(19) United States
(12) Patent Application Publication
(10) Pub. No.: US 2005/0136042 A1
(43) Pub. Date: Jun. 23, 2005

(54) Methods and compositions for tissue repair

(76) Inventors: Oliver B. Betz, Boston, MA (US); Volker M. Betz, Boston, MA (US); Christopher H. Evans, Cohasset, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/917,265

(22) Filed: Aug. 11, 2004

Related U.S. Application Data
(60) Provisional application No. 60/494,484, filed on Aug. 12, 2003.

(51) Int. Cl. A61K 48/00; C12N 5/08; C12N 15/86

(52) U.S. Cl. 424/93.21; 435/455; 435/456; 435/366

(57) ABSTRACT

Methods and compositions for tissue repair are provided. The methods involve obtaining tissue from a patient, including for example, muscle or fat tissue, and contacting the tissue with one or more bioactive agents to induce at least a portion of the cells in the tissue to differentiate into cells of a desired type. The methods and compositions may be used to treat lesions in a variety of tissues, including bone fractures or other injuries.

### RELATED APPLICATIONS

This application claims the benefit of priority to U.S. Provisional Patent Application No. 60/494,484, filed Aug. 12, 2003, which application is hereby incorporated by reference in its entirety.

### BACKGROUND

Tissue repair is the one of the greatest challenges in medicine. Especially in the fields of Orthopaedic Surgery, Plastic and Reconstructive SUrgery and Maxillofacial Surgery, surgeons and scientists are trying to find successful methods to restore bone, cartilage and other tissues.

Massive loss of bone often occurs following trauma (fractures) or from tumor resection (benign tumors and cancer) and everyday it presents major clinical problems to physicians (Wright, V., et al. Molecular Therapy: the Journal of the American Society of Gene Therapy 6(2), 169-78 (2002)). Often the only recourse is an amputation of an extremity due to the lack of structural support (Baltzer, A. W., et al. Gene Therapy 7(9), 734-9 (2000)). Common metabolic diseases like diabetes and osteoporosis more and more frequently cause delayed fracture repair or non-unions even in uncomplicated fractures (Kagel, E. M. & Einhorn, T. A. Iowa Orthop J 16, 147-52 (1996); Kagel, E. M., et al. Current Opinion in Orthopedics 6(5), 7-13 (1995)).

Currently bone defects and non-unions are treated with autologous bone grafts. This method has many disadvantages (limited availability of bone grafts, complicated and extended surgical method, infections paresthesias and pain) and is often unsuccessful (Baltzer A. W., et al. Clinical Orthopaedics & Related Research (379 Suppl), S120-5 (2000a)). Unsatisfactory results after surgical treatment of posttraumatic segmental bone defects are described in up to 30% of cased (Moroni, S., et al. Journal of Epidemiology & Community Health 51(4), 449-52 (1997)).

Biocompatible bone substitutes have been under intensive investigation. These synthetic or natural materials are designed to be osteoconductive and osteoinductive. However, it has been shown that even the most promising material, tri-calcium-phosphate, does not satisfactory induce bone growth but causes inflammation and complete resorption often takes several years (Ignatius, A. A., et al. Journal of Biomedical Materials Research 58(6), 701-9 (2001)). Of further importance is that the production of bone substitutes is expensive.

A very promising new approach to stimulate bone regeneration is the administration of osteoinductive growth factors. It has been shown that such proteins induce or enhance bone formation in vivo (Bostrom, M. et al. Clinical Orthopaedics & Related Research (327), 272-82 (1996); Cook, S. D. & Rueger, D. C. Clinical Orthopaedics & Related Research (324), 29-38 (1996)).

The major drawback of the direct application of these osteoinductive proteins is delivery. They have very short biological half-lives and are difficult to administer in a localized sustained fashion. As a result recombinant proteins have to be delivered at high, unphysiological doses which are very expensive. Under these conditions, the repair site is exposed to initially extremely high concentrations of the osteogenic proteins, followed by a rapid decline. (Bartus, R. T., et al. Science 281(5380), 1161-2 (1998); Bonadio, J., et al. Nat Med 5(7), 753-9 (1999)).

Gene therapy approaches can be used to overcome these problems. Delivery of genes encoding osteoinductive protein products to cell locally at site can provide physiological sustained levels of these factors for extended periods of time (Bonadio, J. Journal of Molecular Medicine 78(6), 303-11 (2000); Goldstein, S. A. Clinical Orthopaedics & Related Research (379 Suppl), S113-9 (2000)).

A second problem for the delivery of osteogenic proteins is the need for scaffolds and matrices. Collagen has been widely used for such purposes, but is expensive to manufacture and is usually recovered from allogenic and xenogeneic sources, leading to a risk of infectious disease and hypersensitivity. Additionally, many times the combination of osteoinductive molecules and collagen still requires the use of bone grafts as a filling material, which causes problems as mentioned above.

It is therefore an object of the present invention to overcome these shortcomings in existing tissue repair techniques, by providing methods and compositions for the preparation of tissue grafts.

### SUMMARY

The present disclosure provides methods and compositions for tissue repair.

In one aspect, a method for producing an activated tissue graft is provided. The method comprises contacting tissue with one or more bioactive agents, wherein the bioactive agents stimulate at least a portion of the cells in the tissue to differentiate into cells of a desired type, thereby producing an activated tissue graft. In an exemplary embodiment, the tissue is contacted with the one or more bioactive agents ex vivo. In another embodiment, the tissue is contacted with the one or more bioactive agents ex situ (e.g., in vivo but at a location different from the site where the tissue being contacted is normally found in the body).

In an exemplary embodiment, the tissue is muscle tissue, such as, for example, skeletal muscle tissue. In another embodiment, the tissue is fat tissue, such as, for example, subcutaneous fat depot.

In certain embodiments, the method may additionally comprise obtaining tissue from a subject.

In various embodiments, the bioactive agents may be one or more of the following: polypeptides, nucleic acids, hormones, cells, drugs, small molecules, and various combinations thereof.

In another aspect, a method for producing an activated tissue graft using a nucleic acid is provided. The method comprises contacting tissue ex vivo or ex situ with at least one nucleic acid encoding at least one bioactive polypeptide thereby introducing said nucleic acid(s) into at least a portion of the cells in said tissue, wherein the bioactive polypeptides are expressed in said cells, and wherein the bioactive polypeptides stimulate at least a portion of the cells in the tissue to differentiate into cells of a desired type, thereby forming an activated tissue graft.

In another aspect, the disclosure provides a composition comprising tissue (such as, for example, muscle or fat tissue) and one or more bioactive agents, wherein the bioactive agents stimulate at least a portion of the cells in the tissue to differentiate into cells of a desired type. In one embodiment, the composition is formed ex vivo or ex situ.

In yet another aspect, the disclosure provides an activated tissue graft comprising tissue which has been exposed to one or more bioactive agents, wherein the bioactive agents stimulate at least a portion of the cells in the tissue to differentiate into cells of a desired type. In one embodiment, the activated tissue graft is exposed to the one or more bioactive agents ex vivo or ex situ.

In another aspect, a method for treating a lesion in a subject is provided. The method comprises (I) contacting tissue with one or more bioactive agents ex vivo or ex situ, wherein the bioactive agents stimulate at least a portion of the cells in the tissue to differentiate into cells of a desired type; and (2) implanting the tissue graft into a lesion of the subject.

In one embodiment, a method for treating a lesion in a subject comprises: (1) contacting tissue with one or more bioactive agents ex vivo or ex situ, wherein the bioactive agents stimulate at least a portion of the cells in the tissue to differentiate into cells of a desired type; (2) implanting the tissue into a location within the subject such that the graft at least partially differentiates into a desired tissue; and (3) transplanting the tissue graft into a lesion of the subject.

In an exemplary embodiment, the disclosure provides a method for producing a bone graft comprising contacting tissue (such as, for example, muscle or fat tissue) with one or more bioactive agents ex vivo or ex situ, wherein the bioactive agents stimulate at least a portion of the cells in the tissue to differentiate into bone cells, thereby producing a bone graft.

In another exemplary embodiment, a method for producing a bone graft using a nucleic acid is provided. The method comprises contacting tissue (such as, for example, muscle or fat tissue) ex vivo or ex situ with at least one nucleic acid encoding at least one bioactive polypeptide thereby introducing said nucleic acid(s) into at least a portion of the cells in said tissue, wherein the bioactive agents are expressed in said cells, and wherein the bioactive agents stimulate at least a portion of the cells in the tissue to differentiate into bone cells, thereby forming a bone graft.

In an exemplary embodiment, the disclosure provides a composition comprising tissue (such as, for example, muscle or fat tissue) and one or more bioactive molecules, wherein the bioactive agents stimulate at least a portion of the cells in the tissue to differentiate into bone cells. In one embodiment, the composition is formed ex vivo or ex situ.

In a further exemplary embodiment, the disclosure provides a bone graft comprising tissue (such as, for example, muscle or fat tissue) which has been exposed to one or more bioactive agents, wherein the bioactive agents stimulate at least a portion of the cells in the tissue to differentiate into bone cells. In one embodiment, the bone graft is formed ex vivo or ex situ.

In another embodiment, methods and compositions for drug delivery are provided. For example, a tissue graft may be contacted with one or more bioactive agents and introduced into a lesion in a subject (for drug delivery at a specified location) or subcutaneously (for release of drug into the blood stream).

In one embodiment, an activated tissue graft may slowly degrade over time and be replaced by ingrowth of the surrounding defect tissue. Ingrowth of the surrounding tissue and/or degradation of the graft may be regulated by gioactive agents that are released by the graft.

In another embodiment, an activated tissue graft may be subjected to mechanical stimulation (e.g., compression or stretching) either ex vivo or in situ to enhance and/or accelerate the transformation of the graft into a desired tissue.

### BRIEF DESCRIPTION OF THE DRAWINGS

FIG. 1 shows X-rays of a rat femur with a critical sized defect (5 mm) that was treated with a muscle graft modified with adenoviruses carrying human BMP-2 cDNA. Panel A shows the femur after surgery, Panel B shows the femur 6 weeks post implantation of the muscle graft, Panel C shows an untransduced muscle graft (control) after 6 weeks, and Panel D shows the histology of the defect area treated with the muscle graft 6 weeks post implantation (Haematoxilin/Eosin staining).

FIG. 2 shows X-rays of a rat femur with a critical sized defect (5 mm) that was treated with a fat graft modified with adenoviruses carrying human BMP-2 cDNA. Panel A shows an untreated defect 8 weeks after surgery, Panel B shows a defect treated with an unmodified fat graft 8 weeks post implantation (control), and Panel C shows a defect treated with a fat graft modified with adenoviruses carrying human BMP-2 cDNA 8 weeks post implantation.

FIG. 3 shows the nucleotide sequence for human bone morphogenetic protein 2 (BMP2) (GenBank Accession No. NM_{—}001200) (SEQ ID NO: 1).

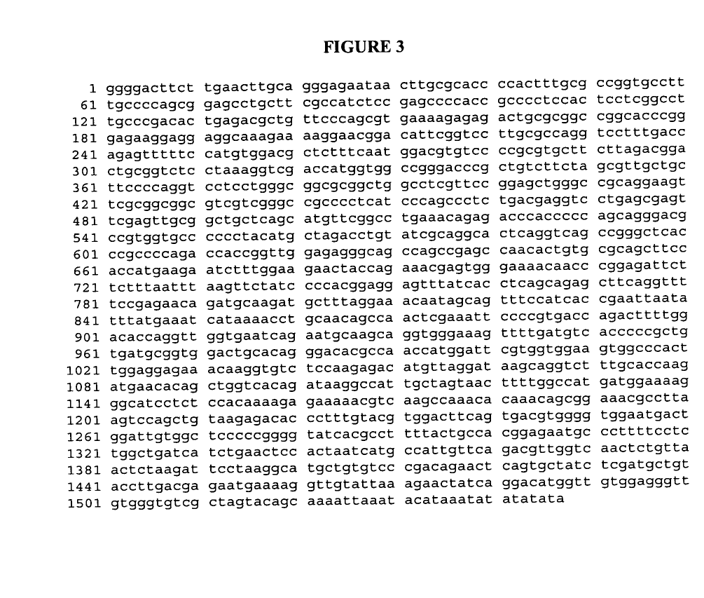

FIG. 4 shows the amino acid sequence for human bone morphogenetic protein 2 (BMP2) (GenBank Accession No. NP_{—}001191) (SEQ ID NO: 2).

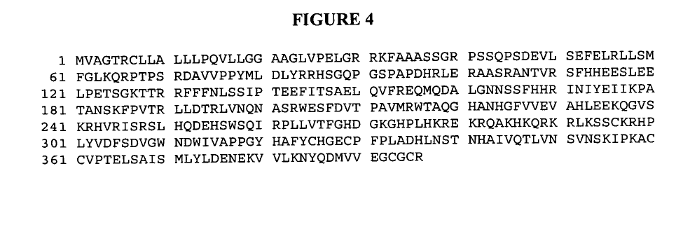

FIG. 5 shows the nucleotide sequence for human bone morphogenetic protein 7 (BMP7) (GenBank Accession No. NM_{—}001719) (SEQ ID NO: 3).

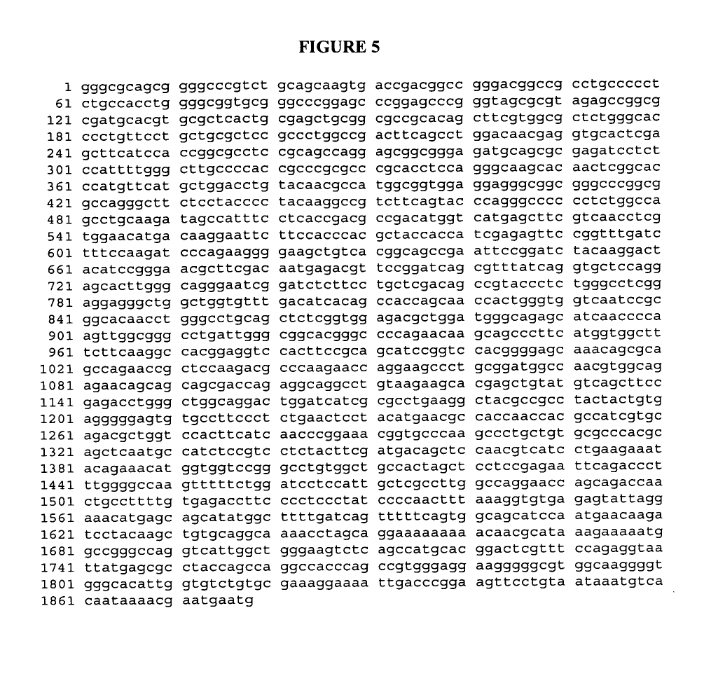

FIG. 6 shows the amino acid sequence for human bone morphogenetic protein 7 (BMP7) (GenBank Accession No. N9_{—}001710) (SEQ ID NO: 4).

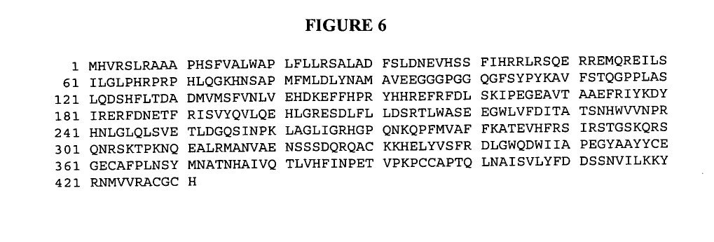

### DETAILED DESCRIPTION

The present disclosure provides methods and compositions for tissue repair. In an exemplary embodiment, novel method for repairing bone lesions are provided. For example, provided are methods and compositions for enhancing the healing of bone defects as they occur in various conditions, such as, severe bone fractures, tumor based bone damages, pseudoarthroses and in the case of loosening prostheses. Further applications include reconstructive and plastic surgery, jawbone surgery and the fusion of vertebrae in the case of severe disc degeneration. Also provided are methods and compositions for the treatment of other musculoskeletal tissues, such as cartilage, intervertebral disc, meniscus, tendon and ligament. In yet other exemplary embodiments, other tissues of the body may be treated.

In various embodiments, this disclosure provides free tissue graft which may be treated to produce a multipotent transplant that transforms into a desired tissue inside or outside of the body. The tissue can be retrieved from almost any site of the body in an ambulant, uncomplicated surgical method that is already standard procedure in plastic and reconstructive surgery. In exemplary embodiments, the tissue graft is obtained from muscle tissue (such as, for example, skeletal muscle tissue) or fat tissue (such as, for example, subcutaneous fat depot).

Modification of the tissue graft may take place either in vivo or ex vivo. The tissue may be genetically modified by viral or non-viral gene transfer methods or altered by exposure to bioactive molecules. In an exemplary embodiment, the modified tissue gradually transforms into the desired tissue inside the patient's body. Alternatively, the transformation of the modified tissue graft may take place outside the patient's body as a tissue engineering method. The tissue graft may be genetically modified by viral or non-viral gene transfer methods or altered by exposure to bioactive molecules in the laboratory. The newly engineered tissue may be transplanted into the defect site of the patient at a variety of time periods, including when the tissue transformation is in the initial stages, when it is nearly complete, or completely transformed, or any time in between.

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “activated tissue graft” refers to tissue which has been contacted with one or more bioactive agents so as to induce at least a portion of the cells in the tissue to differentiate into cells of a desired type. In various embodiments, the term is meant to encompass any tissue which has been so contacted whether or not any of the cells in the graft have actually differentiated into a desired cell type. In exemplary embodiments, at least 1%, 2%, 5%, 10%, 25%, 50%, 75%, 80%, 90%, 95%, 98% or more of the cells in the tissue will differentiate into a desired cell type. Such differentiation may take place ex vivo before implantation of the graft into a subject, after transplantation into a subject, or a combination thereof. A “gene activated tissue graft” refers to tissue which has been contacted with one or more bioactive agents that are nucleic acids, e.g., wherein the nucleic acids encode a product that can induce at least a portion of the cells in the tissue to differentiate into cells of a desired type. A “protein activated tissue graft” refers to tissue which has been contacted with one or more bioactive agents that are polypeptides (or proteins), e.g., wherein the polypeptides (or proteins) can induce at least a portion of the cells in the tissue to differentiate into cells of a desired type. A “cell activated tissue graft” refers to tissue which has been contacted with one or more bioactive agents that are cells, e.g., wherein the cells are progenitor and/or stem cells that may be induced to differentiate into cells of a desired type or wherein the cells have been engineered to express a bioactive polypeptide, etc. An “in vitro transformed tissue graft” refers to tissue which has been contacted with one or more biological agents outside of the subject from which the tissue was obtained. Also provided, are combinations of the above types of activated tissue grafts, including, for example, a gene and protein activated tissue graft, an in vitro transformed protein activated tissue graft, an in vitro transformed gene activated tissue graft, etc. In an exemplary embodiment, an activated tissue graft may be derived from muscle tissue (such as, for example, skeletal muscle tissue) or fat tissue (such as, for example, subcutaneous fat depot). In another embodiment, an activated tissue graft is derived from a tissue other than synovial tissue. The term “activated muscle graft” refers to an activated tissue graft derived from muscle tissue. The term “activated fat graft” refers to an activated tissue graft derived from fat tissue.

The term “BMP-2 nucleic acid” refers to a nucleic acid encoding a BMP-2 polypeptide, e.g., a nucleic acid comprising a sequence consisting of, or consisting essentially of, the polynucleotide sequence set forth in SEQ ID NO: 1. A BMP-2 nucleic acid may comprise all, or a portion of: the nucleotide sequence of SEQ ID NO: 1; a nucleotide sequence at least 60%, 70%, 80%, 90%, 95%, 96%, 97%, 98% or 99% identical to SEQ ID NO: 1; a nucleotide sequence that hybridizes under stringent conditions to SEQ ID NO: 1; nucleotide sequences encoding polypeptides that are functionally equivalent to BMP-2 polypeptides; nucleotide sequences encoding polypeptides at least about 60%, 70%, 80%, 85%, 90%, 95%, 98%, 99% homologous or identical with an amino acid sequence of SEQ ID NO: 2; nucleotide sequences encoding polypeptides having at least one biological activity of a BMP-2 polypeptide and having at least about 60%, 70%, 80%, 85%, 90%, 95%, 98%, 99% homology or identity or more with SEQ ID NO: 2; nucleotide sequences that differ by 1 to about 2, 3, 5, 7, 10, 15, 20, 30, 50, 75 or more nucleotide substitutions, additions or deletions, such as allelic variants, of SEQ ID NO: 1; nucleic acids derived from and evolutionarily related to SEQ ID NO: 1; and complements of, and nucleotide sequences resulting from the degeneracy of the genetic code, for all of the foregoing nd other nucleic acids of the invention. Nucleic acids of the invention also include homologs, e.g., orthologs and paralogs, of SEQ ID NO: 1 and also variants of SEQ ID NO: 1 which have been codon optimized for expression in a particular organism (e.g., host cell).

The term “BMP-7 nucleic acid” refers to a nucleic acid encoding a BMP-7 polypeptide, e.g., a nucleic acid comprising a sequence consisting of, or consisting essentially of, the polynucleotide sequence set forth in SEQ ID NO: 3. A BMP-7 nucleic acid may comprise all, or a portion of: the nucleotide sequence of SEQ ID NO: 3; a nucleotide sequence at least 60%, 70%, 80%, 90%, 95%, 96%, 97%, 98% or 99% identical to SEQ ID NO: 3; a nucleotide sequence that hybridizes under stringent conditions to SEQ ID NO: 3; nucleotide sequences encoding polypeptides that are functionally equivalent to BMP-7 polypeptides; nucleotide sequences encoding polypeptides at least about 60%, 70%, 80%, 85%, 90%, 95%, 98%, 99% homologous or identical with an amino acid sequence of SEQ ID NO: 4; nucleotide sequences encoding polypeptides having at least one biological activity of a BMP-7 polypeptide and having at least about 60%, 70%, 80%, 85%, 90%, 95%, 98%, 99% homology or identity or more with SEQ ID NO: 4; nucleotide sequences that differ by 1 to about 2, 3, 5, 7, 10, 15, 20, 30, 50, 75 or more nucleotide substitutions, additions or deletions, such as allelic variants, of SEQ ID NO: 3; nucleic acids derived from and evolutionarily related to SEQ ID NO: 3; and complements of, and nucleotide sequences resulting from the degeneracy of the genetic code, for all of the foregoing and other nucleic acids of the invention. Nucleic acids of the invention also include homologs, e.g., orthologs and paralogs, of SEQ ID NO: 3 and also variants of SEQ ID NO: 3 which have been codon optimized for expression in a particular organism (e.g., host cell).

The term “BMP-2 polypeptide” refers to a polypeptide comprising the amino acid sequence set forth in SEQ ID NO: 2, or an equivalent or fragment thereof, e.g., a polypeptide comprising a sequence consisting of, or consisting essentially of, the polypeptide sequence set forth in SEQ ID NO: 2. BMP-2 polypeptides include polypeptides comprising all or a portion of the amino acid sequence set forth in SEQ ID NO: 2; the amino acid sequence set forth in SEQ ID NO: 2 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to SEQ ID NO: 2; and functional fragments thereof. BMP-2 polypeptides also include homologs, e.g., orthologs and paralogs, of SEQ ID NO: 2. In exemplary embodiments, a non-naturally occuring BMP-2 polypeptide retains at least one biological activity of a naturally occuring BMP-2 polypeptide. Biological activities of BMP-2 include, for example, the ability to initiate, promote, and/or regulate bone development, growth, remodeling and/or repair. In an exemplary embodiment, a non-naturally occuring BMP-2 polypeptide can induce a muscle or fat cell to differentiate into a cell of a desired type, such as, for example, bone and/or cartilage.

The term “BMP-7 polypeptide” refers to a polypeptide comprising the amino acid sequence set forth in SEQ ID NO: 4, or an equivalent or fragment thereof, e.g., a polypeptide comprising a sequence consisting of, or consisting essentially of, the polypeptide sequence set forth in SEQ ID NO: 4. BMP-7 polypeptides include polypeptides comprising all or a portion of the amino acid sequence set forth in SEQ ID NO: 4; the amino acid sequence set forth in SEQ ID NO: 4 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to SEQ ID NO: 4; and functional fragments thereof. BMP-7 polypeptides also include homologs, e.g., orthologs and paralogs, of SEQ ID NO: 4. In exemplary embodiments, a non-naturally occuring BMP-7 polypeptide retains at least one biological activity of a naturally occuring BMP-7 polypeptide. Biological activities of BMP-7 include, for example, the ability to initiate, promote, and/or regulate bone development, growth, remodeling and/or repair. In an exemplary embodiment, a non-naturally occuring BMP-7 polypeptide can induce a muscle or fat cell to differentiate into a cell of a desired type, such as, for example, bone and/or cartilage.

The term “bioactive agent” refers to a molecule or composition that can induce a muscle or fat cell to differentiate into a cell of a desired type. In various embodiments, a bioactive agent may be one or more of the following: a polypeptide, a polynucleotide, a hormone, a cell, a drug, a small molecule, and various combinations thereof.

The term “bioactive polypeptide” refers to a polypeptide that may be used as a bioactive agent.

The term “conserved residue” refers to an amino acid that is a member of a group of amino acids having certain common properties. The term “conservative amino acid substitution” refers to the substitution (conceptually or otherwise) of an amino acid from one such group with a different amino acid from the same group. A functional way to define common properties between individual amino acids is to analyze the normalized frequencies of amino acid changes between corresponding proteins of homologous organisms (Schulz, G. E. and R. H. Schirmer., Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). One example of a set of amino acid groups defined in this manner include: (i) a charged group, consisting of Glu and Asp, Lys, Arg and His, (ii) a positively-charged group, consisting of Lys, Arg and His, (iii) a negatively-charged group, consisting of Glu and Asp, (iv) an aromatic group, consisting of Phe, Tyr and Trp, (v) a nitrogen ring group, consisting of His and Trp, (vi) a large aliphatic nonpolar group, consisting of Val, Leu and Ile, (vii) a slightly-polar group, consisting of Met and Cys, (viii) a small-residue group, consisting of Ser, Thr, Asp, Asn, Gly, Ala, Glu, Gln and Pro, (ix) an aliphatic group consisting of Val, Leu, Ile, Met and Cys, and (x) a small hydroxyl group consisting of Ser and Thr.

The term “differentiation”, with reference to a cell, refers to a process by which a cell becomes specialized for a particular function or develops a specific phenotype. A “differentiated cell” refers to one that has obtained a fully differentiated state or one that has been programmed for differentiation but is not yet expressing the characteristic phenotype of the specialized cell type which it will eventually become. The term differentiation is also meant to encompass transdifferentiation whereby a cell of one differentiated state changes into a cell with another differentiated state.

The term “naturally-occurring”, as applied to an object, refers to the fact that an object may be found in nature. For example, a polypeptide or polynucleotide sequence that is present in an organism that may be isolated from a source in nature and which has not been intentionally modified by man in the laboratory is naturally-occurring.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “operably linked”, when describing the relationship between two nucleic acid regions, refers to a juxtaposition wherein the regions are in a relationship permitting them to function in their intended manner. For example, a control sequence “operably linked” to a coding sequence is ligated in such a way that expression of the coding sequence is achieved under conditions compatible with the control sequences, such as when the appropriate molecules (e.g., inducers and polymerases) are bound to the control or regulatory sequence(s).

The term “phenotype”, with reference to a cell, refers to the entire physical, biochemical, and physiological makeup of a cell, e.g., having any one trait or any group of traits.

The terms “polypeptide”, “peptide”, and the term “protein”, with reference to a single chain, are used interchangeably herein and refer to a polymer of amino acids. Exemplary polypeptides include gene products, naturally-occurring proteins, homologs, orthologs, paralogs, fragments, and other equivalents, variants and analogs of the foregoing. In certain instances, a protein may comprise two or more polypeptide chains that are associated through covalent or non-covalent interactions.

The terms “polypeptide fragment”, “fragment”, or “truncation polypeptide”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions may occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide.

The term “sequence homology” refers to the proportion of base matches between two nucleic acid sequences or the proportion of amino acid matches between two amino acid sequences. When sequence homology is expressed as a percentage, e.g., 50%, the percentage denotes the proportion of matches over the length of sequence from a desired sequence that is compared to some other sequence. Gaps (in either of the two sequences) are permitted to maximize matching; gap lengths of 15 bases or less are usually used, 6 bases or less are used more frequently, with 2 bases or less used even more frequently. The term “sequence identity” means that sequences are identical (i.e., on a nucleotide-by-nucleotide basis for nucleic acids or amino acid-by-amino acid basis for polypeptides) over a window of comparison. The term “percentage of sequence identity” is calculated by comparing two optimally aligned sequences over the comparison window, determining the number of positions at which the identical amino acids occurs in both sequences to yield the number of matched positions, dividing the number of matched positions by the total number of positions in the comparison window, and multiplying the result by 100 to yield the percentage of sequence identity. Methods to calculate sequence identity are known to those of skill in the art and described in further detail below.

The term “specifically hybridizes” refers to detectable and specific nucleic acid binding. Polynucleotides, oligonucleotides and nucleic acids of the invention selectively hybridize to nucleic acid strands under hybridization and wash conditions that minimize appreciable amounts of detectable binding to nonspecific nucleic acids. Stringent conditions may be used to achieve selective hybridization conditions as known in the art and discussed herein. Generally, the nucleic acid sequence homology between the polynucleotides, oligonucleotides, and nucleic acids of the invention and a nucleic acid sequence of interest will be at least 30%, 40%, 50%, 60%, 70%, 80%, 85%, 90%, 95%, 98%, 99%, or more. In certain instances, hybridization and ashing conditions are performed under stringent conditions according to conventional hybridization procedures and as described further herein.

The terms “stringent conditions” or “stringent hybridization conditions” refer to conditions which promote specific hydribization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of the a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature.

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a prehybridization step may be conducted prior to hybridization. Prehybridization may be carried out for at least about 1 hour, 3 hours or 10 hours in the same solution and at the same temperature as the hybridization solution (without the complementary polynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y; S. Agrawal (ed.) Methods in Molecular Biology, volume 20; Tijssen (1993) Laboratory Techniques in biochemistry and molecular biology-hybridization with nucleic acid probes, e.g., part 1 chapter 2 “Overview of principles of hybridization and the strategy of nucleic acid probe assays”, Elsevier, New York; and Tibanyenda, N. et al., Eur. J. Biochem. 139:19 (1984) and Ebel, S. et al., Biochem. 31:12083 (1992).

A “subject” is essentially any organism, although usually a vertebrate, and most typically a mammal, such as a human or a non-human mammal.

As used herein, the term “tissue” refers to an aggregation of similarly specialized cells united in the performance of a particular function. Tissue is intended to encompass all types of biological tissue including both hard and soft tissue, including connective tissue (e.g., hard forms such as osseous tissue or bone) as well as other muscular or skeletal tissue.

The term “vector” refers to a nucleic acid capable of transporting another nucleic acid to which it has been linked. One type of vector which may be used herein is an episome, i.e., a nucleic acid capable of extra-chromosomal replication. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA molecules that, in their vector form are not bound to the chromosome. In the present specification, “plasmid” and “vector” are used interchangeably as the plasmid is the most commonly used form of vector. However, the present disclosure is intended to include such other forms of expression vectors which serve equivalent functions and which become known in the art subsequently hereto.

Tissue

The methods and compositions described herein utilize tissue for formation of an activated tissue graft that may be used for the treatment of lesions in a variety of tissue types in a subject.

In one embodiment, activated tissue grafts may comprise muscle tissue. Any type of muscle tissue may be used including skeletal muscle, cardiac muscle and/or smooth muscle tissue. In exemplary embodiments, skeletal muscle tissue from any appropriate source in the body is used. Exemplary sources of skeletal muscle tissue include the back, neck, and chest, including the pectoralis major, rectus abdominis, diaphragm, trapezius, and latissimus dorsi; the shoulder and arm, including the deltoid, triceps brachii, and biceps brachii; and the leg and ankle, including the gluteus maximus, sartorius, quadriceps femoris, gracilis, hamstrings, biceps femoris, semitendinosus, gastrocnemius, and achilles tendon. In exemplary embodiments, muscle tissue is obtained from a source that is easily accessible, which minimizes the invasiveness of the procedure to obtain the tissue, and which minimizes the injury to site from which the tissue was obtained. Exemplary muscle tissue sources include muscles of the thigh, calf, bicep and forearm.

In another embodiment, activated tissue grafts may comrpise fat tissue. Any type of fat tissue may be utilized, including subcutaneous depots from such areas as the chest, abdomen and buttocks, hips and waist. Visceral fat depots may aslo be used, such as that found above the kidneys.

Tissue may be extracted from a subject using methods standard in the art for obtaining tissue for grafting. For example, such tissue may be surgically extracted using standard or minimally invasive surgical techniques. Minimal-invasive surgery (MIS) refers to surgical procedures using surgical and diagnostic instruments specially developed to reduce the amount of physical trauma associated with the procedure. Generally, MIS involves instruments that may be passed through natural or surgically created openings of small diameter into a body to a desired location of use so that surgical intervention is possible with substantially less stress being imposed on the patient, for example, without general anesthesia. MIS may be accomplished using visualization methods such as fiberoptic or microscopic means. Examples of MIS include, for example, arthoscopic surgery, laparoscopic surgery, endoscopic surgery, thoracic surgery, neurosurgery, bladder surgery, gastrointestinal tract surgery, etc.

In certain embodiments, tissue is removed from a subject in a size and shape suitable for implantation into a specific lesion. In other embodiments, the tissue is removed from the subject and then is altered to a desired size and shape ex vivo using standard techniques. Methods for shaping grafts are described, for example, in U.S. Pat. No. 6,503,277.

In certain embodiments, the tissue for use in treatment of a subject may be autologous (obtained from the recipient), allogeneic (obtained from a donor subject other than the recipient), xenogenic (obtained from a different species, e.g., a non-human donor, such as, for example, a pig). For allogeneic sources, the closest possible immunological match between donor and recipient is desired. If an autologous source is not available or warranted, donor and recipient Class I and Class II histocompatibility antigens can be analyzed to determine the closest match available. This minimizes or eliminates immune rejection and reduces the need for immunosuppressive or immunomodulatory therapy. If required, immunosuppressive or immunomodulatory therapy can be started before, during, and/or after the graft is introduced into a patient. For example, cyclosporin A, or other immunosuppressive drugs, can be administered to the recipient. Immunological tolerance may also be induced prior to transplantation by alternative methods known in the art (D. J. Watt et al., 1984, Clin. Exp. Immunol. 55:419; D. Faustman et al., 1991, Science 252:1701). In exemplary embodiments, autologous grafts are used.

Appropriate sterile conditions may be used when extracting, handling and implanting the grafts. Such sterile techniques will be known to the skilled artisan based on the teachings herein.

Formation of Activated Grafts

Activated tissue grafts may be formed by contacting tissue (such as, for example, muscle or fat tissue) with one or more bioactive agents. In various embodiments suitable bioactive agents include, for example, polypeptides, nucleic acids, cells, small molecules, hormones, and combinations thereof. Contacting the graft with the bioactive agents may involve soaking and/or coating the graft with a solution or composition comprising one or more bioactive agents. Coating the graft with an appropriate solution or composition may be carried out using a brush or spatula, by spraying the solution onto the surface of the graft, or by dipping the graft into the solution or composition. Contacting the graft with the bioactive agents may also involve injecting or infusing the graft with a solution or composition comprising one or more bioactive agents. In certain embodiments, combinations of these or other methods may be used. When injection is used for application of the bioactive agents, multiple injections at different locations within the tissue may be used.

The tissue may be contacted with the one or more bioactive agents at various time points. For example, the tissue may be contacted with the bioactive agents at one or more of the following times: prior to removal of the tissue from a patient, after removal of the tissue from a patient, concurrently with implanting the tissue in the same or a different patient, and/or after implantation into the same or a different patient. In an exemplary embodiments, the tissue is contacted with one or more bioactive agents ex vivo, e.g., after the tissue has been removed from a patient and prior to implantation into the same or a different patient. In various embodiments, tissue may be contacted with one or more bioactive agents on one or more occasions, for example, at least one, two, three, four, five, six, seven, eight, nine, ten, or more applications of the bioactive agent may be made to the tissue. Such applications may be made at one time (e.g., with the span of an hour) or over a period of time, for example, over at least two hours, five hours, ten hours, 24 hours, two days, three days, four days, five days, one week, two weeks, three weeks, one month, or more.

In certain embodiments, the graft may be incubated ex vivo before implantation into a patient. In exemplary embodiments, the graft is incubated ex vivo after application of the bioactive agents and before implantation into a patient. Such incubation period may be for at least 1 min, 1 hour, 2 hours, 5 hours, 10 hours 24, hours, 2 days, 5 days, 1 week, 2 weeks, or longer. During incubation, the graft may be stored under conditions which minimize damage or degradation of the graft. In one embodiment, the graft is incubated ex vivo for a period of time sufficient to allow at least a portion of the cells in the tissue graft to differentiate, or initiate differentiation, into a desired cell type.

In another embodiment, agents may also be used to control the growth and function of cells contained within or surrounding the graft after implantation, including, for example, the ingrowth of blood vessels and/or the deposition and organization of fibrous tissue around the graft. For example, agents such as growth factors, angiogenic factors, compounds selectively inhibiting ingrowth of fibroblast tissue such as anti-inflammatories, and compounds selectively inhibiting growth and proliferation of transformed (cancerous) cells may be utilized in accordance with this embodiment of the disclosure. In another embodiment, agents may be used to attract or promote ingrowth of pluripotent stem cells and/or progenitor cells into the tissue graft. After migration into the tissue graft, bioactive agents may be used to stimulate the differentiation of the stem cells and/or progenitor cells into cells of a desired type.

In another embodiment, tissue may be contacted with one or more agents that increase the response of the tissue to a bioactive agent. For example, tissue may be contacted with an agent that increases the level of a receptor of a bioactive agent, such as, for example, a BMP-2 or BMP-7 receptor.

Bioactive Agents

Bioactive agents suitable for use in the methods and compositions described herein may be polypeptides, polynucleotides, hormones, cells, drugs and/or small molecules, and various combinations thereof.

In certain embodiments, bioactive agents may be mixed with or encapsulated in a substance that facilitates its delivery to and/or uptake by a cell. In one embodiment, polynucleotides are mixed with cationic lipids that are useful for the introduction of nucleic acid into the cell, including, but not limited to, LIPOFECTIN™ (DOTMA) which consists of a monocationic choline head group that is attached to diacylglycerol (see generally, U.S. Pat. No. 5,208,036 to Epstein et al.); TRANSFECTAM™ (DOGS) a synthetic cationic lipid with lipospermine head groups (Promega, Madison, Wis.); DMRIE and DMRIE.HP (Vical, La Jolla, Calif.); DOTAP™ (Boehringer Mannheim (Indianapolis, Ind.), and Lipofectamine (DOSPA) (Life Technology, Inc., Gaithersburg, Md.).

In other embodiments, bioactive agents may be mixed with or encapsulated into microspheres or nanospheres that promote penetration into mammalian tissues and uptake by mammalian cells. In various embodiments, the microspheres or nanospheres may optionally have other molecules bound to them. These modifications may, for example, impart the microspheres or nanospheres with the ability to target and bind specific tissues or cells, allow them be retained at the administration site, protect incorporated bioactive agents, exhibit antithrombogenic effects, prevent aggregation, and/or alter the release properties of the microspheres. Production of such surface-modified microspheres are discussed in Levy et al., PCT Application No. WO 96/20698, the disclosure of which is hereby incorporated by reference. In exemplary embodiments, it may be desirable to incorporate receptor-specific molecules into or onto the microspheres to mediate receptor-specific particle uptake, including, for example, antibodies such as IgM, IgG, IgA, IgD, and the like, or any portions or subsets thereof, cell factors, cell surface receptors, MHC or HLA markers, viral envelope proteins, peptides or small organic ligands, derivatives thereof, and the like.

In other embodiments, bioactive agents may be mixed or complexed with particulates that promote delivery to, or uptake by mammalian cells, provide osteoconductive properties, influence mass transport, etc. In certain embodiments, suitable particulates include bioceramics such as hydroxyapatite (“HA”) or other calcium containing compounds such as mono-, di-, octa-, alpha-tri-, beta-tri-, or tetra-calcium phosphate, fluoroapatite, calcium sulfate, calcium fluoride and mixtures thereof; bioactive glass comprising metal oxides such as calcium oxide, silicon dioxide, sodium oxide, phosphorus pentoxide, and mixtures thereof; and the like. In an exemplary embodiment, hydroxyapatite is used as the bioceramic material because it provides osteoinductive and/or osteoconductive properties. It is preferable that the particle size of the particulates be about 0.1 nm to about 100 nm, more preferably about 2 nm to about 50 nm.

In various embodiments, bioactive agents may be formulated so as to provide controlled release over time, for example, days, weeks, months or years. This may be accomplished by encapsulating the bioactive agents in microspheres. Release properties may be determined by the size and physical characteristics of the microspheres.

Also provided are bioactive agents formulated in pharmaceutical compositions. Such compositions typically will comprise therapeutically effective amounts of one or more bioactive agents, i.e., an amount sufficient to cause or facilitate at least a portion of the cells in a tissue graft to differentiate into cells of a desired tissue type. For example, dosage levels of between about 0.01 and about 100 mg/kg body, or between about 0.5 and about 75 mg/kg, may be used in accordance with the methods and compositions disclosed herein. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound. The pharmaceutical compositions may also contain a pharmaceutically acceptable excipient. Such excipients include any pharmaceutical agent which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

Polypeptides

In certain embodiments, bioactive agents may be polypeptides or biologically active derivatives or fragments of polypeptides. Any polypeptides which cause, stimulate or facilitate at least a portion of the cells in a tissue graft (such as, for example, muscle tissue or fat tissue) to differentiate into cells of a desired tissue type may be used as bioactive agents. Suitable polypeptides which may be used as bioactive agents include, for example, growth factors, cytokines, morphogenesis factors, cell signalling factors, cell differentiation factors, polypeptides which stimulate or suppress cell division, and polypeptides which modulate the rate of cell division.

In exemplary embodiments, polypeptides which may be used as bioactive agents include, for example, osteoinductive, angiogenic, mitogenic, or similar substances, such as transforming growth factors (TGFs), for example, TGF-alpha, TGF-beta-1, TGF-beta-2, TGF-beta-3; fibroblast growth factors (FGFs), for example, acidic and basic fibroblast growth factors (aFGF and bFGF); platelet derived growth factors (PDGFs); platelet-derived endothelial cell growth factor (PD-ECGF); PDGF-BB; enamel matrix derivative (EMD); amelogenin like factors, connective tissue growth factors (CTGF); scleraxis; Osterix; Runx-2; Runx-3; AML; Cbfa-1, Notch-1; PPAR gama; Menin; Smad-1, Smad-2, Smad-3, Smad-4, Smad-5; LIF; FGF-2; tumor necrosis factor alpha (TNF-alpha); tumor necrosis factor beta (TNF-b); epidermal growth factors (EGFs); connective tissue activated peptides (CTAPs); osteogenic factors, for example, BMP-1, BMP-2, BMP-3, BMP-4, BMP-5, BMP-6, BMP-7, BMP-8, BMP-9, BMP-10, BMP-11, BMP-12, BMP-13, BMP-15, BMP-16; LIM mineralization proteins (LMPs), for example, LMP-1, LMP-2, LMP-3; cartilage-derived morphogenetic proteins (CDMP), for example, DCMP-1, CDP-2, CDMP-3; cartilage growth factor (CGF); insulin-like growth factor (IGF), for example, IGF-I and IGF-II; erythropoietin; heparin binding growth factor (hbgf); vascular endothelium growth factor (VEGF); hepatocyte growth factor (HGF); colony stimulating factor (CSF); macrophage-CSF (M-CSF); granulocyte/macrophage CSF (GM-CSF); nitric oxide synthase (NOS); nerve growth factor (NGF); brain derived nerve growth factor (BDNF); brain derived growth factor (BDGF); glial derived neurotrophic factor (GDNF); NICD (an oncogenic form of Notch); BMP/RA-inducible neural-specific proteins (BRINP); neurotrophin; neurotrophin-3; cardiotrophin-1; forskolin; ciliary neurotrophic factor; muscle morphogenic factor (MMP); Inhibins (for example, Inhibin A, Inhibin B); growth differentiating factors (for example, GDF-1); Activins (for example, Activin A, Activin B, Activin AB); angiogenin; angiotensin; angiopoietin; angiotropin; antiangiogenic antithrombin (aaAT); atrial natriuretic factor (ANF); betacellulin; endostatin; endothelial cell-derived growth factor (ECDGF); endothelial cell growth factor (ECGF); endothelial cell growth inhibitor; endothelial monocyte activating polypeptide (EMAP); endothelial cell-viability maintaining factor; endothelin (ET); endothelioma derived mobility factor (EDMF); heart derived inhibitor of vascular cell proliferation; hematopoietic growth factors; erythropoietin (Epo); interferon (IFN); interleukins (IL); oncostatin M; placental growth factor (PlGF); somatostatin; transferrin; thrombospondin; vasoactive intestinal peptide; Indian Hedgehog (IHH); sonic hedgehog (Shh); Wnt; Dickkopf-1; parathyroid hormone related protein (PTHRP); Sry-type high-mobility group (HMG) box proteins (SOX) transcription factors, for example, Sox-5, Sox-6, Sox-9; and biologically active analogs, fragments, and derivatives of such polypeptides.

In one embodiment, the bioactive agents are one or more Osteogenic proteins such as the Bone Morphogenetic Proteins (BMPs). Exemplary BMPs include, for example, BMP-2, BMP-3, BMP-4, BMP-5, BMP-6 and BMP-7, disclosed for instance in U.S. Pat. Nos. 5,108,922; 5,013,649; 5,116,738; 5,106,748; 5,187,076, 5,459,047, 5,849,880; and 5,141,905; BMP-8, disclosed in PCT publication WO 91/18098; and BMP-9, disclosed in PCT publication WO 93/00432, BMP-10, disclosed in PCT application WO 94/26893; BMP-11, disclosed in PCT application WO 94/26892, or BMP-12 or BMP-13, disclosed in PCT application WO 95/16035, or BMP-15, disclosed in PCT application WO 96/36710 or BMP-16, disclosed in U.S. Pat. No. 5,965,403. In an exemplary embodiment the bioactive agent is BMP-2, BMP-7, or a combination thereof. Other examplary osteogenic factors include, for example, Osterix, Runx-2, Cbfa-1, AML, sonic hedgehog, indian hedgehog, Menin, Notch-1, and PPAR gama.

Examples of chondrogenic factors include, for example, Runx-3, Smad-1, Smad-2, Smad-3, Smad-4, Smad-5, Sox-5, and Sox-6.

Examples of differentiation factors for mesenchymal stem cells include, for example, LIF, FGF-2, HGF, Wnt, and Dickkopf-1.

Examples of neurogenic factors include, for example, BDGF, GDNF, NICD, bFGF, BRINP, neurotrophin-3, cardiotrophin-1, forskolin, and ciliary neurotrophic factor.

Examplex of growth factors that may be useful in association with ligament formation include, for example, PDGF-BB, PDGF, EMD, Amelogenin like factors, CTGF, and scleraxis.

In certain embodiments, polypeptides used as bioactive agents may be purified from an organism which naturally expresses the polypeptide or from an organism which has been engineered to express a recombinant form of the polypeptide. In certain embodiments, polypeptides that may be used in accordance with the methods and compositions described herein may be modified so that its rate of traversing the cellular membrane is increased. For example, the polypeptide may be fused to a second peptide which promotes “transcytosis,” e.g., uptake of the peptide by cells. The peptide may be a portion of the HIV transactivator (TAT) protein, such as the fragment corresponding to residues 37-62 or 48-60 of TAT, portions which have been observed to be rapidly taken up by a cell in vitro (Green and Loewenstein, (1989) Cell 55:1179-1188). Alternatively, the internalizing peptide may be derived from the Drosophila antennapedia protein, or homologs thereof. The 60 amino acid long homeodomain of the homeo-protein antennapedia has been demonstrated to translocate through biological membranes and can facilitate the translocation of heterologous polypeptides to which it is coupled. Thus, polypeptides may be fused to a peptide consisting of about amino acids 42-58 of Drosophila antennapedia or shorter fragments for transcytosis (Derossi et al. (1996) J Biol Chem 271:18188-18193; Derossi et al. (1994) J Biol Chem 269:10444-10450; and Perez et al. (1992) J Cell Sci 102:717-722). The transcytosis polypeptide may also be a non-naturally-occurring membrane-translocating sequence (MTS), such as the peptide sequences disclosed in U.S. Pat. No. 6,248,558.

In certain embodiments, polypeptides used as bioactive agents may be synthesized chemically, in a cell free system, or within a cell. Chemical synthesis of polypeptides may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and T. W. Muir et al., Curr. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Schnolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

In other embodiments, polypeptides used as bioactive agents may be purchased from a commercial supplier. Numerous commercial suppliers sell a wide variety of polypeptides that may be used as bioactive agents, including, for example, Pierce Biotechnology Inc. (Rockford, Ill., www.piercenet.com), Cytoshop (Tel-Aviv, Israel, www.cytoshop.com), PromoCell GmbH (Heidelberg, Germany, www.promokine.de), Research Diagnostics, Inc. (Flanders, N.J., www.researchd.com), and PeproTech Inc. (Rocky Hill, N.J., www.peprotech.com). For example, human BMP-2 may be purschased from PeproTech Inc. (Cat. No. 120-O₂), Research Diagnostics, Inc. (Cat. No. RDI-1202), or PromoCell GmbH (Cat. No. C-67310) and BMP-7 may purchased from PeproTech Inc. (Cat. No. 120-03).

Nucleic Acids

In certain embodiments, bioactive agents may be polynucleotides, or fragments thereof. Examples of polynucleotides which are useful as bioactive agents include, but are not limited to, DNA, RNA, cDNA and recombinant nucleic acids; naked DNA, cDNA, and RNA; genomic DNA, cDNA or RNA; oligonucleotides; aptomeric oligonucleotides; ribozymes; anti-sense oligonucleotides (including RNA or DNA); DNA coding for an anti-sense RNA; DNA coding for tRNA or rRNA molecules (i.e., to replace defective or deficient endogenous molecules); double stranded small interfering RNAs (siRNAs); polynucleotide peptide bonded oligos (PNAs); circular or linear RNA; circular single-stranded DNA; self-replicating RNAs; mRNA transcripts; catalytic RNAs, including, for example, hammerheads, hairpins, hepatitis delta virus, and group I introns which may specifically target and/or cleave specific RNA sequences in vivo; polynucleotides coding for therapeutic proteins or polypeptides, as further defined herein; chimeric nucleic acids, including, for example, DNA/DNA hybrids, RNA/RNA hybrids, DNA/RNA hybrids, DNA/peptide hybrids, and RNA/peptide hybrids; DNA compacting agents; and gene/vector systems (i.e., any vehicle that allows for the uptake and expression of nucleic acids), including nucleic acids in a non-infectious vector (i.e., a plasmid) and nucleic acids in a viral vector. In an exemplary embodiment, chimeric nucleic acids, include, for example, nucleic acids attached to a peptide targeting sequences that directs the location of the chimeric molecule to a location within a body, within a cell, or across a cellular membrane (i.e., a membrane translocating sequence (“MTS”)). In another embodiment, a nucleic acid may be fused to a constitutive housekeeping gene, or a fragment thereof, which is expressed in a wide variety of cell types.

In exemplary embodiments, polynucleotides which are useful as bioactive agents are polynucleotides comprising a nucleotide sequence encoding a polypeptide as described above, such as, a polypeptide which causes, stimulates or facilitates at least a portion of the cells in a tissue graft (such as, for example, muscle tissue or fat tissue) to differentiate into cells of a desired tissue type. Further examples of suitable polynucleotides include those that encode growth factors, cytokines, morphogenesis factors, cell signaling factors, cell differentiation factors, polypeptides which stimulate or suppress cell division, and polypeptides which modulate the rate of cell division.

In another exemplary embodiment, polynucleotides which are useful as bioactive agents are polynucleotides comprising a nucleotide sequence encoding one or more of the following polypeptides: transforming growth factors (TGFs), for example, TGF-alpha, TGF-beta-1, TGF-beta-2, TGF-beta-3; fibroblast growth factors (FGFs), for example, acidic and basic fibroblast growth factors (aFGF and bFGF); platelet derived growth factors (PDGFs); platelet-derived endothelial cell growth factor (PD-ECGF); PDGF-BB; enamel matrix derivative (EMD); amelogenin like factors, connective tissue growth factors (CTGF); scleraxis; Osterix; Runx-2; Runx-3; AML; Cbfa-1, Notch-1; PPAR gama; Menin; Smad-1, Smad-2, Smad-3, Smad-4, Smad-5; LIF; FGF-2; tumor necrosis factor alpha (TNF-alpha); tumor necrosis factor beta (TNF-b); epidermal growth factors (EGFs); connective tissue activated peptides (CTAPs); osteogenic factors, for example, BMP-1, BMP-2, BMP-3, BMP-4, BMP-5, BMP-6, BMP-7, BMP-8, BMP-9, BMP-10, BMP-1, BMP-12, BMP-13, BMP-15, BMP-16; LIM mineralization proteins (LMPs), for example, LMP-1, LMP-2, LMP-3; cartilage-derived morphogenetic proteins (CDMP), for example, DCMP-1, CDP-2, CDMP-3; cartilage growth factor (CGF); insulin-like growth factor (IGF), for example, IGF-I and IGF-II; erythropoietin; heparin binding growth factor (hbgf); vascular endothelium growth factor (VEGF); hepatocyte growth factor (HGF); colony stimulating factor (CSF); macrophage-CSF (M-CSF); granulocyte/macrophage CSF (GM-CSF); nitric oxide synthase (NOS); nerve growth factor (NGF); brain derived nerve growth factor (BDNF); brain derived growth factor (BDGF); glial derived neurotrophic factor (GDNF); NICD (an oncogenic form of Notch); BMP/RA-inducible neural-specific proteins (BRINP); neurotrophin; neurotrophin-3; cardiotrophin-1; forskolin; ciliary neurotrophic factor; muscle morphogenic factor (MMP); Inhibins (for example, Inhibin A, Inhibin B); growth differentiating factors (for example, GDF-1); Activins (for example, Activin A, Activin B, Activin AB); angiogenin; angiotensin; angiopoietin; angiotropin; antiangiogenic antithrombin (aaAT); atrial natriuretic factor (ANF); betacellulin; endostatin; endothelial cell-derived growth factor (ECDGF); endothelial cell growth factor (ECGF); endothelial cell growth inhibitor; endothelial monocyte activating polypeptide (EMAP); endothelial cell-viability maintaining factor; endothelin (ET); endothelioma derived mobility factor (EDMF); heart derived inhibitor of vascular cell proliferation; hematopoietic growth factors; erythropoietin (Epo); interferon (IFN); interleukins (IL); oncostatin M; placental growth factor (PlGF); somatostatin; transferrin; thrombospondin; vasoactive intestinal peptide; Indian Hedgehog (IHH); sonic hedgehog (Shh); Wnt; Dickkopf-1; parathyroid hormone related protein (PTHRP); Sry-type high-mobility group (HMG) box proteins (SOX) transcription factors, for example, Sox-5, Sox-6, Sox-9; and biologically active analogs, fragments, and derivatives of such polypeptides.

A nucleic acid encoding a polypeptide suitable for use as a bioactive agent may be obtained from mRNA or genomic DNA from any organism in accordance with protocols generally known to those skilled in the art. A cDNA encoding a polypeptide suitable for use as a bioactive agent, for example, may be obtained by isolating total mRNA from an organism, e.g. a human, etc. Double stranded cDNAs may then be prepared from the total mRNA, and subsequently inserted into a suitable plasmid or bacteriophage vector using any one of a number of known techniques. A gene encoding a polypeptide suitable for use as a bioactive agent may also be cloned using established polymerase chain reaction (PCR) techniques in accordance with the nucleotide sequence information provided herein or as available from publicly accessible databases. PCR refers to a procedure or technique in which target nucleic acid is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. Using PCR, a nucleic acid sequence can be amplified from RNA or DNA. For example, a nucleic acid sequence can be isolated by PCR amplification from total cellular RNA, total genomic DNA, and cDNA as well as from bacteriophage sequences, plasmid sequences, viral sequences, and the like. When using RNA as a source of template, reverse transcriptase can be used to synthesize complimentary DNA strands. General procedures for PCR are taught in MacPherson et al., PCR: A PRACTICAL APPROACH, (IRL Press at Oxford University Press, (1991)). PCR conditions may be empirically determined based on a number of parameters which influence the reaction. Among these parameters are annealing temperature and time, extension time, Mg²⁺ and ATP concentration, pH, and the relative concentration of primers, templates and deoxyribonucleotides. Appropriate primers and reaction conditions may be readily determined by the skilled artisan based on the teachings herein. After amplification, the resulting fragments can be detected by agarose gel electrophoresis followed by visualization with ethidium bromide staining and ultraviolet illumination.

Polynucleotides suitable for use as a bioactive agent may also be produced in part or in total by chemical synthesis, e.g., by the phosphoramidite method described by Beaucage and Carruthers, Tetra. Letts., 22:1859-1862 (1981) or the triester method according to the method described by Matteucci et al., J. Am. Chem. Soc., 103:3185 (1981), and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

In certain embodiments, polynucleotides useful as bioactive agents may be modified so as to increase resistance to nucleases, e.g. exonucleases and/or endonucleases, and therefore have increased stability in vivo. Exemplary modifications include, but are not limited to, phosphoramidate, phosphothioate and methylphosphonate analogs of nucleic acids (see also U.S. Pat. Nos. 5,176,996; 5,264,564; and 5,256,775).

In certain embodiments, polynucleotides useful as a bioactive agent may be contained within a vector. Vectors suitable for use herein, include, viral vectors or vectors derived from viral sources, for example, adenoviral vectors, herpes simplex vectors, papilloma vectors, adeno-associated vectors, retroviral vectors, pseudorabies virus, alpha-herpes virus vectors, and the like. A thorough review of viral vectors, particularly viral vectors suitable for modifying nonreplicating cells, and how to use such vectors in conjunction with the expression of polynucleotides of interest can be found in the book Viral Vectors: Gene Therapy and Neuroscience Applications Ed. Caplitt and Loewy, Academic Press, San Diego (1995). In other embodiments, vectors may be non-infectious vectors, or plasmids. Suitable non-infectious vectors, include, but are not limited to, mammalian expression vectors that contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17.

In certain embodiments, vectors which may be used in accordance with the methods and compositions of the invention are those in which the coding portion of the DNA segment, whether encoding a full length protein or smaller peptide, is operably linked to at least one transcriptional regulatory sequence. Regulatory sequences are art-recognized and are selected to direct expression of the subject proteins in the desired fashion (time and place). Transcriptional regulatory sequences are described in Goeddel; Gene Expression Technology: Methods in Enzymology 185, Academic Press, San Diego, Calif. (1990). Exemplary regulatory sequences include, for example, promoters and enhancer elements. A “promoter” refers to a DNA sequence recognized by the synthetic machinery inherent to, or introduced into, the cell which facilitates initiation of the specific transcription of a gene.

The promoter may be in the form of the promoter that is naturally associated with a gene encoding a polypeptide useful as a bioactive agent, as may be obtained by isolating the 5′ non-coding sequences located upstream of the coding segment or exon, for example, using recombinant cloning and/or PCR technology. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

In exemplary embodiments, a promoter is employed that directs the expression of the DNA segment in a specific cell and/or tissue type. The use of promoter and cell type combinations for protein expression is generally known to those of skill in the art of molecular biology, for example, see Sambrook et al. (1989), incorporated herein by reference. The promoters employed may be constitutive, or inducible, and can be used under the appropriate conditions to direct high level expression of the introduced DNA segment if desired.

In an exemplary embodiment, nucleic acid constructs comprise promoters and/or enhancers that direct transcription of genes that are specific for, or highly expressed in, cells found in muscle tissue, particularly skeletal muscle tissue. Such promoter and enhancer elements may be referred to as muscle specific regulatory elements. A muscle specific regulatory element is any regulatory element which affects the transcription or expression of a gene specifically in muscle tissue and not in other body tissues (see e.g., U.S. Pat. No. 6,310,196). A muscle specific regulatory element may be a muscle specific promoter, but it may also include one or more enhancers. Examples of muscle specific regulatory elements include those which are isolated from muscle specific genes, such as the muscle isozyme of creatine kinase (MCK) (Sternberg et al., 1988), myosin light kinase (Merlie 1992a, 1992b), muscle-specific aldolase (Concordet et al., 1993), muscle-specific enolase (Gaillongo et al., 1993), troponin C (Prigozy et al., 1993), myosin (Kitsis et at., 1991; Takeda et al., 1992, von Harsdorf et al., 1993). Many of these promoters are under the control of the MyoD family of transcription factors (Olsen 1990; Hart 1992). These regulatory elements, as well as other muscle specific regulatory elements, may be modified to remove unnecessary sequences as long as they retain the muscle specificity of action.

In another embodiment, nucleic acid constructs comprise promoters and/or enhancers that direct transcription of genes that are specific for, or highly expressed in, cells found in fat tissue, particularly subcutaneous fat depot. Such promoter and enhancer elements may be referred to as fat specific regulatory elements. A fat specific regulatory element is any regulatory element which affects the transcription or expression of a gene specifically in fat tissue and not in other body tissues. A fat specific regulatory element may be a fat specific promoter, but it may also include one or more enhancers, such as an adipose tissue specific element (ASE). Examples of fat specific regulatory elements include those which are isolated from fat specific genes, glucose transporter 4 (GLUT4; Miura et al., Biochem. Biophys. Res. Commun. (2003) 312:277-284), adiponectin (Seo et al., J. Biol. Chem. (2004) 279:22108-17), hormone sensitive lipase (HSL; Smih et al., Diabetes (2002) 51:293-300) perilipin (Nagai et al., Endocrinology (2004) 145:2346-56). These regulatory elements, as well as other fat specific regulatory elements, may be modified to remove unnecessary sequences as long as they retain the muscle specificity of action.

Additionally any suitable promoter/enhancer combination (as per the Eukaryotic Promoter Data Base EPDB) may also be used to drive expression. In one embodiment, use of a T3, T7 or SP6 cytoplasmic expression system is contemplated. Eukaryotic cells can support cytoplasmic transcription from certain bacterial promoters if the appropriate bacterial polymerase is provided, either as part of the delivery complex or as an additional genetic expression construct.

In certain embodiments, constitutive promoter, such as a strong viral promoter (e.g., a CMV promoter) may be used. Alternatively, the promoter may be an inducible promoter, e.g., a metallothionein promoter. Other inducible promoters include those that are controlled by the inducible binding, or activation, of a transcription factor, e.g., as described in U.S. Pat. Nos. 5,869,337 and 5,830,462 by Crabtree et al., describing small molecule inducible gene expression (a genetic switch); International patent applications WO 94/18317, WO 96/06111, WO 96/41865 and the like, as well as in other heterologous transcription systems such as those involving tetracyclin-based regulation reported by Bujard et al., generally referred to as an allosteric “off-switch” described by Gossen and Bujard (Proc. Natl. Acad. Sci. U.S.A. (1992) 89:5547) and in U.S. Pat. Nos. 5,464,758; 5,650,298; and 5,589,362 by Bujard et al. Other inducible transcription systems involve steroid or other hormone-based regulation.

In certain embodiments, a bioactive agent may comprise two or more polypeptides or proteins. For example, two or more polypeptides may be co-expressed in the same cell or a gene may be provided to a cell that already has another selected protein. Co-expression may be achieved by co-transfecting the cell with two distinct recombinant vectors, each bearing a copy of either of the respective DNAs. Alternatively, a single recombinant vector may be constructed to include the coding regions for both of the proteins, which could then be expressed in cells transfected with the single vector.

Any means for the introduction of polynucleotides into mammals, human or non-human, may be used in accordance with the methods described herein for the delivery of the polynucleotide constructs into a tissue graft. In an exemplary method of the invention, the DNA constructs are delivered using an expression vector. The expression vector may be a viral vector or a plasmid that harbors the polynucleotide. Nonlimiting examples of viral vectors useful according to this aspect of the invention include lentivirus vectors, herpes simplex virus vectors, adenovirus vectors, adeno-associated virus vectors, various suitable retroviral vectors, pseudorabies virus vectors, alpha-herpes virus vectors, HIV-derived vectors, other neurotropic viral vectors and the like. In another embodiment of the invention, the DNA constructs are delivered to cells by transfection, i.e., by delivery of “naked” DNA or in a complex with a colloidal dispersion system.

Gene delivery vehicles useful in the practice of the present invention can be constructed utilizing methodologies well known in the art of molecular biology, virology, microbiology, molecular biology and recombinant DNA techniques within the skill of the art. Such techniques are explained fully in the literature.

Adeno-Associated Vectors. An exemplary viral vector system useful for delivery of the subject polynucleotides is the adeno-associated virus (AAV). Human adenoviruses are double-stranded DNA viruses which enter cells by receptor-mediated endocytosis. These viruses have been considered well suited for gene transfer because they are easy to grow and manipulate and they exhibit a broad host range in vivo and in vitro. Adenoviruses are able to infect quiescent as well as replicating target cells and persist extrachromosomally, rather than integrating into the host genome. AAV is a helper-dependent DNA parvovirus which belongs to the genus Dependovirus. AAV has no known pathologies and is incapable of replication without additional helper functions provided by another virus, such as an adenovirus, vaccinia or a herpes virus, for efficient replication and a productive life cycle. In the absence of the helper virus, AAV establishes a latent state by insertion of its genome into a host cell chromosome. Subsequent infection by a helper virus rescues the integrated copy which can then replicate to produce infectious viral progeny. The combination of the wild type AAV virus and the helper functions from either adenovirus or herpes virus generates a recombinant AVV (rAVV) that is capable of replication. One advantage of this system is its relative safety (For a review, see Xiao et al., (1997) Exp. Neurol. 144:113-124).

The AAV genome is composed of a linear, single-stranded DNA molecule which contains approximately 4681 bases (Berns and Bohenzky, (1987) Advances in Virus Research (Academic Press, Inc.) 32:243-307). The genome includes inverted terminal repeats (ITRs) at each end which function in cis as origins of DNA replication and as packaging signals for the virus. The internal nonrepeated portion of the genome includes two large open reading frames, known as the AAV rep and cap regions, respectively. These regions code for the viral proteins involved in replication and packaging of the virion. For a detailed description of the AAV genome, see, e.g., Muzyczka, N. (1992) Current Topics in Microbiol. and Immunol. 158:97-129.

Vectors containing as little as 300 base pairs of AAV can be packaged and can integrate. Space for exogenous DNA is limited to about 4.7 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

AAV has not been associated with the cause of any disease. AAV is not a transforming or oncogenic virus. AAV integration into chromosomes of human cell lines does not cause any significant alteration in the growth properties or morphological characteristics of the cells. These properties of AAV also recommend it as a potentially useful human gene therapy vector. AAV vectors are capable of transducing both dividing and non-dividing cells in vitro and in vivo (Afione, S. A., et al., (1996), J. Virol. 70:3235-3241; Flotte, T. R., et al., (1993), Pro. Natl. Acad. Sci USA 90: 10613-10617; Flotte, T., R., (1994), Am. J. Respir. Cell Mol. Biol. 11:517-521; Kaplitt, M. G., et al., (1994), Nat. Genet. 8:148-154; Kaplitt, M. G., et al., (1996), Ann. Thoracic Surg. 62:1669-1676; McCown, T. J., et al., (1996), Brain Res. 713:99-107; Muzyczka, N. (1992), Curr. Top. Microbiol. Immunol. 158: 97-129; Podsakoff, G., et al., (1994), J. Virol. 68: 5656-5666; Russell, D. W., et al., (1994), Proc. Natl. Acad. Sci USA 91:8915-8919; Ziao, X., et al., (1996), J. Virol., 70:8098-8108). An example of a high frequency of successful integration of AAV DNA into non-dividing cells is the transduction of pulmonary epithelial cells (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973).

General methods for the construction and delivery of rAAV constructs are well known in the art and may be found in Barlett, J. S., et al., (1996), Protocols for Gene Transfer in Neuroscience; Towards Gene Therapy of Neurological Disorders, pp. 115-127. The AAV-based expression vector to be used typically includes the 145 nucleotide AAV inverted terminal repeats (ITRs) flanking a restriction site that can be used for subcloning of a desired nucleotide sequence, either directly using the restriction site available, or by excision of the desired nucleotide sequence with restriction enzymes followed by blunting of the ends, ligation of appropriate DNA linkers, restriction digestion, and ligation into the site between the ITRs. The capacity of AAV vectors is about 4.4 kb. The following proteins have been expressed using various AAV-based vectors, and a variety of promoter/enhancers: neomycin phosphotransferase, chloramphenicol acetyl transferase, Fanconi's anemia gene, cystic fibrosis transmembrane conductance regulator, and granulocyte macrophage colony-stimulating factor (Kotin, R. M., Human Gene Therapy 5:793-801, 1994, Table I). As an alternative to inclusion of a constitutive promoter such as CMV to drive expression of the polynucleotide of interest, an AAV promoter can be used (ITR itself or AAV p5 (Flotte, et al. J. Biol. Chem. 268:3781-3790, 1993)).

AAV is also capable of infecting a broad variety of host cells without triggering pathogenic or inflammatory side effects.(Wu et al., (1998) J. Virol. 72(7):5919-26; Xiao et al., (1997) Exp. Neurol. 144:113-124, W7, W21, W28).

Production & Packaging of Adeno-Associated Vectors. Polynucleotides may be inserted into vector genomes using methods known in the art based on the teachings herein. For example, insert and vector DNA can be contacted, under suitable conditions, with a restriction enzyme to create complementary or blunt ends on each molecule that can pair with each other and be joined with a ligase. Alternatively, synthetic nucleic acid linkers can be ligated to the termini of a polynucleotide. These synthetic linkers can contain nucleic acid sequences that correspond to a particular restriction site in the vector DNA. Other means are known and available in the art.

In an exemplary embodiment, the viral vectors are AAV vectors. By an “AAV vector” is meant a vector derived from an adeno-associated virus serotype, including without limitation, AAV-1, AAV-2, AAV-3, AAV-4, AAV-5, AAVX7, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector typically includes at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

In one embodiment, AAV expression vectors are constructed using known techniques to provide as operatively linked components in the direction of transcription, control elements including a transcriptional initiation region, the DNA of interest and a transcriptional termination region. The control elements are selected to be functional in a mammalian cell. The resulting construct which contains the operatively linked components is bounded (5′ and 3′) with functional AAV ITR sequences.

An AAV expression vector which harbors a DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. For example, ligations can be accomplished in 20 mM Tris-Cl pH 7.5, 10 mM MgCl.sub.2, 10 mM DTT, 33 μg/ml BSA, 10 mM-50 mM NaCl, and either 40 uM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226.

Additionally, heterologous genes can be produced synthetically to include AAV ITR sequences arranged 5′ and 3′ of one or more selected nucleic acid sequences. The complete heterologous sequence is assembled from overlapping oligonucleotides prepared by standard methods. See, e.g., Edge, Nature (1981) 292:756; Nambair et al. Science (1984) 223:1299; Jay et al. J. Biol. Chem. (1984) 259:6311.

Methods for in vitro packaging AAV vectors are also available and have the advantage that there is no size limitation of the DNA packaged into the particles (see, U.S. Pat. No. 5,688,676, by Zhou et al., issued Nov. 18, 1997). This procedure involves the preparation of cell free packaging extracts.

Production of rAAV Virions. A vector comprising transcriptional regulatory elements and a gene of interest can be packaged into AAV virions. For example, a human cell line such as, for example, 293 can be co-transfected with the AAV-based expression vector and another plasmid containing open reading frames encoding AAV Rep and Cap genes under the control of endogenous AAV promoters or a heterologous promoter. In the absence of helper virus, the rep proteins Rep68 and Rep78 prevent accumulation of the replicative form, but upon superinfection with adenovirus or herpes virus, these proteins permit replication from the ITRs (present only in the construct containing the desired nucleotide sequence) and expression of the viral capsid proteins. This system results in packaging of the desired nucleotide sequence into AAV virions (Carter, B. J., Current Opinion in Biotechnology 3:533-539, 1992; Kotin, R. M, Human Gene Therapy 5:793-801, 1994; Bartlett, J. S., et al., (1996), Towards Gene Therapy of Neurological Disorders, pp. 115-127; Flotte, T. R., et al., (1995), Gene Ther. 2:29-37; Samulski, R. J., et al., (1989), J. Virol. 63: 3822-3828; Snyder, R., et al., (1996), Current Protocols in Human Genetics, pp 12.1.1-12.2.23). Typically, about three days after transfection, recombinant AAV is harvested from the cells along with adenovirus and the contaminating adenovirus is then inactivated by heat treatment. In another embodiment, packaging can be accomplished through the use of an engineered AAV packaging cell line and an AAV producer cell line where the AAV helper plasmid has been transfected into a human cell line (Clark, K. R., et al., (1995) Hum. Gene Ther. 6: 1329-1341).

Methods to improve the titer of AAV can also be used to package a polynucleotide into an AAV virion. Such strategies include, but are not limited to: stable expression of the ITR-flanked nucleotide sequence in a cell line followed by transfection with a second plasmid to direct viral packaging; use of a cell line that expresses AAV proteins inducibly, such as temperature-sensitive inducible expression or pharmacologically inducible expression. Alternatively, a cell can be transformed with a first AAV vector including a 5′ ITR, a 3′ ITR flanking a heterologous gene, and a second AAV vector which includes an inducible origin of replication, e.g., SV40 origin of replication, which is capable of being induced by an agent, such as the SV40 T antigen and which includes DNA sequences encoding the AAV rep and cap proteins. Upon induction by an agent, the second AAV vector may replicate to a high copy number, and thereby increased numbers of infectious AAV particles may be generated (see, e.g, U.S. Pat. No. 5,693,531 by Chiorini et al., issued Dec. 2, 1997). In yet another method for producing large amounts of recombinant AAV, a chimeric plasmid is used which incorporates the Epstein Barr Nuclear Antigen (EBNA) gene, the latent origin of replication of Epstein Barr virus (orip) and an AAV genome. These plasmids are maintained as multicopy extra-chromosomal elements in cells. Upon addition of wild-type helper functions, these cells will produce high amounts of recombinant AAV (U.S. Pat. No. 5,691,176 by Lebkowski et al., issued Nov. 25, 1997). In another system, an AAV packaging plasmid is provided that allows expression of the rep gene, wherein the p5 promoter, which normally controls rep expression, is replaced with a heterologous promoter (U.S. Pat. No. 5,658,776, by Flotte et al., issued Aug. 19, 1997). Additionally, one may increase the efficiency of AAV transduction by treating the cells with an agent that facilitates the conversion of the single stranded form to the double stranded form, as described in Wilson et al., WO 96/39530.

AAV stocks can be produced as described in Hermonat and Muzyczka (1984) PNAS 81:6466, modified by using the pAAV/Ad described by Samulski et al. (1989) J. Virol. 63:3822. Concentration and purification of the virus can be achieved by reported methods such as banding in cesium chloride gradients, as was used for the initial report of AAV vector expression in vivo (Flotte, et al. J. Biol. Chem. 268:3781-3790, 1993) or chromatographic purification, as described in O'Riordan et al., WO 97/08298.

In order to produce rAAV virions, an AAV expression vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

For the purposes of the invention, suitable host cells for producing rAAV virions include microorganisms, yeast cells, insect cells, and mammalian cells, that can be, or have been, used as recipients of a heterologous DNA molecule. Cells from the stable human cell line, 293 (readily available through, e.g., the American Type Culture Collection under Accession Number ATCC CRL1573) are exemplary in the practice of the present invention. Particularly, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a convenient platform in which to produce rAAV virions.

AAV Helper Functions & AAV Accessory Functions. Host cells containing the above-described AAV expression vectors may be rendered capable of providing AAV helper functions to facilitate replication and encapsidation of the bioactive agent nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof.

AAV helper functions may be introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection.

Both AAV expression vectors and AAV helper constructs can be constructed to contain one or more optional selectable markers. Suitable markers include genes which confer antibiotic resistance or sensitivity to, impart color to, or change the antigenic characteristics of those cells which have been transfected with a nucleic acid construct containing the selectable marker when the cells are grown in an appropriate selective medium. Exemplary selectable marker genes that are useful in the practice of the invention include, for example, the hygromycin B resistance gene (encoding Aminoglycoside phosphotranferase (APH)) that allows selection in mammalian cells by conferring resistance to G418 (available from Sigma, St. Louis, Mo.). Other suitable markers will be known to those of skill in the art based on the teachings herein.

In certain embodiments, the host cell (or packaging cell) may be rendered capable of providing non AAV derived functions, or “accessory functions,” in order to facilitate the production of rAAV virions. Particularly, accessory functions can be introduced into and then expressed in host cells using methods known to those of skill in the art. Commonly, accessory functions are provided by infection of the host cells with an unrelated helper virus. A number of suitable helper viruses are known, including adenoviruses; herpesviruses such as herpes simplex virus types 1 and 2; and vaccinia viruses. Nonviral accessory functions will also find use herein, such as those provided by cell synchronization using any of various known a gents. S ee, e.g., Buller et al. (1981) J. Virol. 40:241-247; McPherson et al. (1985) Virology 147:217-222; Schlehofer et al. (1986) Virology 152:110-117.

Alternatively, accessory functions can be provided using an accessory function vector. Accessory function vectors include nucleotide sequences that provide one or more accessory functions. An accessory function vector is capable of being introduced into a suitable host cell in order to support efficient AAV virion production in the host cell. Accessory function vectors can be in the form of a plasmid, phage, transposon or cosmid. Accessory vectors can also be in the form of one or more linearized DNA or RNA fragments which, when associated with the appropriate control elements and enzymes, can be transcribed or expressed in a host cell to provide accessory functions. See, for example, WO 97/17458.

Nucleic acid sequences providing the accessory functions can be obtained from natural sources, such as from the genome of an adenovirus particle, or constructed using recombinant or synthetic methods known in the art. In this regard, adenovirus-derived accessory functions have been widely studied, and a number of adenovirus genes involved in accessory functions have been identified and partially characterized. See, e.g., Carter, B. J. (1990) “Adeno-Associated Virus Helper Functions,” in CRC Handbook of Parvoviruses, vol. I (P. Tijssen, ed.), and Muzyczka, N. (1992) Curr. Topics. Microbiol. and Immun. 158:97-129. Specifically, early adenoviral gene regions E1a, E2a, E4, VAI RNA and, possibly, E1b are thought to participate in the accessory process. Janik et al. (1981) Proc. Natl. Acad. Sci. USA 78:1925-1929. Herpesvirus-derived accessory functions have been described. See, e.g., Young et al. (1979) Prog. Med. Virol. 25:113. Vaccinia virus-derived accessory functions have also been described. See, e.g., Carter, B. J. (1990), supra., Schlehofer et al. (1986) Virology 152:110-117.

As a consequence of the infection of the host cell with a helper virus, or transfection of the host cell with an accessory function vector, accessory functions are expressed which transactivate the AAV helper construct to produce AAV Rep and/or Cap proteins. The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions.

Following recombinant AAV replication, rAAV virions can be purified from the host cell using a variety of conventional purification methods, such as CsCl gradients. Further, if infection is employed to express the accessory functions, residual helper virus can be inactivated, using known methods. For example, adenovirus can be inactivated by heating to temperatures of approximately 60° C. for, e.g., 20 minutes or more. This treatment effectively inactivates only the helper virus since AAV is extremely heat stable while the helper adenovirus is heat labile.

The resulting rAAV virions are then ready for use for DNA delivery to a muscle or fat tissue graft in accordance with the methods and compositions described herein.

rAAV Vector as a Non-Viral Delivery Vector. An alternative delivery option with rAAV vectors is to uncouple the integration episome properties from the viral component and to combine it with a non-viral delivery vehicle. In an exemplary embodiment the non-viral delivery vehicle is a liposome. (Baudard, M., et al., (1996), Hum. Gene Ther. 7: 1309-1322; During, M., et al., (1996), Soc. Neurosci. Abstr. 18.12; Philip, R., et al., (1994), Mol. Cell. Biol. 14: 2411-2418) Philip et al, have demonstrated the use of the rAAV-liposome combination in primary T-lymphocytes and primary and cultured tumor cells. (Philip, R., et al., (1994), Mol. Cell. Biol. 14: 2411-2418). In that study, cell transfection resulted in sustained expression of the IL-2 gene. A similar methodology was also employed to in the treatment of Canavan's disease (During, M., et al., (1996), Soc. Neurosci. Abstr. 18.12). In vivo delivery of the rAAV-liposome combination has also been demonstrated. Baudard et al. have shown that sustained expression may be maintained in the mouse following an in vivo delivery of the complex through the tail of the mouse (Baudard, M., et al., (1996), Hum. Gene Ther. 7: 1309-1322). In vivo delivery targeted to the central nervous system has been demonstrated by Wu et al., who achieved neuropeptide Y gene expression in the neocortex and the hypothalamic paraventricular nucleus of the brain following the injection of Sendai virosomes complexed with an rAAV plasmid. (Wu. P., et al., (1996) Gene Ther. 3: 246-253).

For additional detailed guidance on AAV technology which may be useful in the practice of the subject invention, including methods and materials for the incorporation of a nucleotide sequence, the propagation and purification of the recombinant AAV vector containing the nucleotide sequence, and its use in transfecting cells and mammals, see e.g. Carter et al, U.S. Pat. No. 4,797,368 (10 Jan. 1989); Muzyczka et al, U.S. Pat. No. 5,139,941 (18 Aug. 1992); Lebkowski et al, U.S. Pat. No. 5,173,414 (22 Dec. 1992); Srivastava, U.S. Pat. No. 5,252,479 (12 Oct. 1993); Lebkowski et al, U.S. Pat. No. 5,354,678 (11 Oct. 1994); Shenk et al, U.S. Pat. No. 5,436,146(25 Jul. 1995); Chatterjee et al, U.S. Pat. No. 5,454,935 (12 Dec. 1995), Carter et al WO 93/24641 (published 9 Dec. 1993), and Natsoulis, U.S. Pat. No. 5,622,856 (Apr. 22, 1997). Further information regarding AAVs and the adenovirus or herpes helper functions required can be found in the following articles: Berns and Bohensky (1987), “Adeno-Associated Viruses: An Update”, Advanced-in Virus Research, Academic Press, 33:243-306. The genome of AAV is described in Laughlin et al. (1983) “Cloning of infectious adeno-associated virus genomes in bacterial plasmids”, Gene, 23: 65-73. Expression of AAV is described in Beaton et al. (1989) “Expression from the Adeno-associated virus p5 and p19 promoters is negatively regulated in trans by the rep protein”, J. Virol., 63:4450-4454. Construction of rAAV is described in a number of publications: Tratschin et al. (1984) “Adeno-associated virus vector for high frequency integration, expression and rescue of genes in mammalian cells”, Mol. Cell. Biol., 4:2072-2081; Hermonat and Muzyczka (1984) “Use of adeno-associated virus as a mammalian DNA cloning vector: Transduction of neomycin resistance into mammalian tissue culture cells”, Proc. Natl. Acad. Sci. USA, 81:6466-6470; McLaughlin et al. (1988) “Adeno-associated virus general transduction vectors: Analysis of Proviral Structures”, J. Virol., 62:1963-1973; and Samulski et al. (1989) “Helper-free stocks of recombinant adeno-associated viruses: normal integration does not require viral gene expression”, J. Virol., 63:3822-3828. Cell lines that can be transformed by rAAV are those described in Lebkowski et al. (1988) “Adeno-associated virus: a vector system for efficient introduction and integration of DNA into a variety of mammalian cell types”, Mol. Cell. Biol., 8:3988-3996. “Producer” or “packaging” cell lines used in manufacturing recombinant retroviruses are described in Dougherty et al. (1989) J. Virol., 63:3209-3212; and Markowitz et al. (1988) J. Virol., 62:1120-1124.

Adenoviral Vectors. In certain embodiments, a viral gene delivery system useful in the present invention utilizes adenovirus-derived vectors. Knowledge of the genetic organization of adenovirus, a 36 kB, linear and double-stranded DNA virus, allows substitution of a large piece of adenoviral DNA with foreign sequences up to 8 kB. The infection of adenoviral DNA into host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. Recombinant adenovirus is capable of transducing both dividing and non-dividing cells. The ability to effectively transduce non-dividing cells makes adenovirus a good candidate for gene transfer into muscle or fat cells.

Adenovirus is particularly suitable for use as a gene transfer vector because of its mid-sized genome, ease of manipulation, high titer, wide target-cell range, and high infectivity. Both ends of the viral genome contain 100-200 base pair (bp) inverted terminal repeats (ITR), which are cis elements necessary for viral DNA replication and packaging. The early (E) and late (L) regions of the genome contain different transcription units that are divided by the onset of viral DNA replication. The E1 region (EIA and EIB) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan (1990) Radiotherap. Oncol. 19:197). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and all the mRNAs issued from this promoter possess a 5′ tripartite leader (TL) sequence which makes them exemplary mRNAs for translation.

The genome of an adenovirus can be manipulated such that it encodes a gene product of interest, but is inactivated in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584; Ragot et al. (1993) Nature 361:647).

Furthermore, the virus particle is relatively stable and amenable to purification and concentration, and as above, can be modified so as to affect the spectrum of infectivity. Additionally, adenovirus is easy to grow and manipulate and exhibits broad host range in vitro and in vivo. This group of viruses can be obtained in high titers, e.g., 10⁹-10¹¹ plaque-forming unit (PFU)/ml, and they are highly infective. The life cycle of adenovirus does not require integration into the host cell genome. The foreign genes delivered by adenovirus vectors are episomal, and therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors. Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted polynucleotide of the invention can be under control of, for example, the E1A promoter, the major late promoter (MLP) and associated leader sequences, the viral E3 promoter, or exogenously added promoter sequences.

In certain embodiments, the adenovirus vector may be replication defective, or conditionally defective. The adenovirus may be of any of the 42 different known serotypes or subgroups A-F. Adenovirus type 5 of subgroup C is the exemplary starting material in order to obtain the conditional replication-defective adenovirus vector for use in accordance with the methods and compositions described herein. This is because Adenovirus type 5 is a human adenovirus about which a great deal of biochemical and genetic information is known, and it has historically been used for most constructions employing adenovirus as a vector. As stated above, the typical vector according to the present invention is replication defective and will not have an adenovirus E1 region. Thus, it will be most convenient to introduce the nucleic acid of interest at the position from which the E1 coding sequences have been removed. However, the position of insertion of the polynucleotide in a region within the adenovirus sequences is not critical to the present invention. For example, it may also be inserted in lieu of the deleted E3 region in E3 replacement vectors as described previously by Karlsson et. al. (1986) or in the E4 region where a helper cell line or helper virus complements the E4 defect.

An exemplary helper cell line is 293 (ATCC Accession No. CRL1573). This helper cell line, also termed a “packaging cell line” was developed by Frank Graham (Graham et al. (1987) J. Gen. Virol. 36:59-72 and Graham (1977) J. General Virology 68:937-940) and provides E1A and E1B in trans. However, helper cell lines may also be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

Adenoviruses can also be cell type specific, i.e., infect only restricted types of cells and/or express a desired nucleotide sequence only in restricted types of cells. For example, the viruses may comprise a gene under the transcriptional control of a transcription initiation region specifically regulated by target host cells, as described e.g., in U.S. Pat. No. 5,698,443. Thus, expression from replication competent adenoviruses can be restricted to certain cells by, e.g., inserting a cell specific response element to regulate synthesis of a protein necessary for replication, e.g., E1A or E1B.

DNA sequences of a number of adenovirus types are available from Genbank. For example, human adenovirus type 5 has GenBank Accession No. M73260. The adenovirus DNA sequences may be obtained from any of the 42 human adenovirus types currently identified. Various adenovirus strains are available from the American Type Culture Collection, Rockville, Md., or by request from a number of commercial and academic sources. A polynucleotide as described herein may be incorporated into any a denoviral vector and delivery protocol, by restriction digest, linker ligation or filling in of ends, and ligation.

Adenovirus producer cell lines can include one or more of the adenoviral genes E1, E2a, and E4 DNA sequence, for packaging adenovirus vectors in which one or more of these genes have been mutated or deleted have been described in the literature. See, e.g., WO 96/18418 by Kadan et al.; WO 95/346671 by Kovesdi et al.; WO 94/28152 by Imler et al.; WO 95/02697 by Pyrrocaudet et al.; and WO 96/14061 by Wang et al.

Hybrid Adenovirus-AAV Vectors. In certan embodiments, a hybrid adenovirus-AAV vector may be used in accordance with the methods and compositions described herein. Hybrid Adenovirus-AAV vectors comprise an adenovirus capsid containing a nucleic acid having a portion of an adenovirus, and 5′ and 3′ ITR sequences from an AAV which flank a selected nucleotide sequence under the control of a promoter. See e.g. Wilson et al, International Patent Application Publication No. WO 96/13598. This hybrid vector is characterized by high titer delivery of a nucleotide sequence to a host cell and the ability to stably integrate the nucleotide sequence into the host cell chromosome in the presence of the rep gene. This virus is capable of infecting virtually all cell types (conferred by its adenovirus sequences) and stable long term integration of the nucleotide sequence into the host cell genome (conferred by its AAV sequences).

The adenovirus nucleic acid sequences employed in this vector can range from a minimum sequence amount, which requires the use of a helper virus to produce the hybrid virus particle, to only selected deletions of adenovirus genes, which deleted gene products can be supplied in the hybrid viral process by a packaging cell. For example, a hybrid virus can comprise the 5′ and 3′ inverted terminal repeat (ITR) sequences of an adenovirus (which function as origins of replication). The left terminal sequence (5′) sequence of the Ad5 genome that can be used spans bp 1 to about 360 of the conventional adenovirus genome (also referred to as map units 0-1) and includes the 5′ ITR and the packaging/enhancer domain. The 3′ adenovirus sequences of the hybrid virus include the right terminal 3′ ITR sequence which is about 580 nucleotides (about bp 35,353—end of the adenovirus, referred to as about map units 98.4-100).

The AAV sequences useful in the hybrid vector are viral sequences from which the rep and cap polypeptide encoding sequences are deleted and are usually the cis acting 5′ and 3′ ITR sequences. Thus, the AAV ITR sequences are flanked by the selected adenovirus sequences and the AAV ITR sequences themselves flank a selected nucleotide sequence. The preparation of the hybrid vector is further described in detail in published PCT application entitled “Hybrid Adenovirus-AAV Virus and Method of Use Thereof”, WO 96/13598 by Wilson et al.

For additional detailed guidance on adenovirus and hybrid adenovirus-AAV technology which may be useful in the practice of the subject invention, including methods and materials for the incorporation of a nucleotide sequence, the propagation and purification of recombinant virus containing the nucleotide sequence, and its use in transfecting cells and mammals, see also Wilson et al, WO 94/28938, WO 96/13597 and WO 96/26285, and references cited therein.

Retroviruses. In certain embodiments, retroviral vectors may be used in accordance with the methods and compositions described herein. The retroviruses are a group of single-stranded RNA viruses characterized by an ability to convert their RNA to double-stranded DNA in infected cells by a process of reverse-transcription (Coffin (1990) Retroviriae and their Replication” In Fields, Knipe ed. Virology. New York: Raven Press). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene, termed psi, functions as a signal for packaging of the genome into virions. Two long terminal repeat (LTR) sequences are present at the 5′ and 3′ ends of the viral genome. These contain strong promoter and enhancer sequences and are also required for integration in the host cell genome (Coffin (1990), supra).

In order to construct a retroviral vector, a nucleic acid of interest is inserted into the viral genome in the place of certain viral sequences to produce a virus that is replication-defective. In order to produce virions, a packaging cell line containing the gag, pol, and env genes but without the LTR and psi components is constructed (Mann et al. (1983) Cell 33:153). When a recombinant plasmid containing a human cDNA, together with the retroviral LTR and psi sequences is introduced into this cell line (by calcium phosphate precipitation for example), the psi sequence allows the RNA transcript of the recombinant plasmid to be packaged into viral particles, which are then secreted into the culture media (Nicolas and Rubenstein (1988) “Retroviral Vectors”, In: Rodriguez and Denhardt ed. Vectors: A Survey of Molecular Cloning Vectors and their Uses. Stoneham, Butterworth, and Temin, (1986) “Retrovirus Vectors for Gene Transfer: Efficient Integration into and Expression of Exogenous DNA in Vertebrate Cell Genome”, In: Kucherlapati ed. Gene Transfer. New York: Plenum Press; Mann et al., 1983, supra). The media containing the recombinant retroviruses is then collected, optionally concentrated, and used for gene transfer. Retroviral vectors are able to infect a broad variety of cell types.

The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a protein of the present invention, e.g., a transcriptional activator, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. An exemplary retroviral vector is a pSR MSVtkNeo (Muller et al. (1991) Mol. Cell Biol. 11:1785 and pSR MSV(XbaI) (Sawyers et al. (1995) J. Exp. Med. 181:307) and derivatives thereof. For example, the unique BamHI sites in both of these vectors can be removed by digesting the vectors with BamHI, filling in with Klenow and religating to produce pSMTN2 and pSMTX2, respectively, as described in WO 96/41865 by Clackson et al. Examples of suitable packaging virus lines for preparing both ecotropic and amphotropic retroviral systems include Crip and Cre.

Retroviruses, including lentiviruses, have been used to introduce a variety of genes into many different cell types, including neural cells, epithelial cells, retinal cells, endothelial cells, lymphocytes, myoblasts, hepatocytes, bone marrow cells, in vitro and/or in vivo (see for example, review by Federico (1999) Curr. Opin. Biotechnol. 10:448; Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO 93/25234, WO 94/06920, and WO 94/11524). For instance, strategies for the modification of the infection spectrum of retroviral vectors include coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, and can also be used to convert an ecotropic vector in to an amphotropic vector.

Other Viral Systems. Other viral vector systems that can be used to deliver nucleic acid may be derived from, for example, herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth, Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. Exemplary viruses include, for example, an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al.(1990) J. Virol., 64:642-650).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990, supra). This suggested that large portions of the genome could be replaced with foreign genetic material. The hepatotropism and persistence (integration) were particularly attractive properties for liver-directed gene transfer. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al. (1991) Hepatology, 14:124A).

Non-viral Transfer. Several non-viral methods for the transfer of expression constructs into mammalian cells are contemplated by the present invention. These include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat'l Acad. Sci. USA, 81:7161-7165, 1984), direct microinjection, DNA-loaded liposomes (Nicolau and Sene, Biochim. Biophys. Acta, 721:185-190, 1982; Fraley et al., Proc. Nat'l Acad. Sci. USA, 76:3348-3352, 1979), cell sonication (Fechheimer et al., Proc. Nat'l Acad. Sci. USA, 84:8463-8467, 1987), gene bombardment using high velocity microprojectiles (Yang et al., Proc. Nat'l Acad. Sci USA, 87:9568-9572, 1990), receptor-mediated transfection (Wu and Wu, J. Biol. Chem., 2 62:4429-4432, 19877; Wu and Wu, Biochem., 27:887-892, 1988). In other embodiments, transfer of nucleic acids into cells may be accomplished by formulating the nucleic acids with nanocaps (e.g., nanoparticulate CaPO₄), colloidal gold, nanoparticulate synthetic polymers, and/or liposomes. In an exemplary embodiment, polynucleotides may be associated with QDOT™ Probes (www.qdots.com).

Once the construct has been delivered into the cell the nucleic acid encoding the polypeptide may be positioned and expressed at different sites. In certain embodiments, the nucleic acid encoding the polypeptide may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, (Wu G, Wu C ed.), New York: Marcel Dekker, pp. 87-104, 1991). The addition of DNA to cationic liposomes causes a topological transition from liposomes to optically birefringent liquid-crystalline condensed globules (Radler et al., Science, 275:810-814, 1997). These DNA-lipid complexes are potential non-viral vectors for use in gene therapy.

Liposome-mediated nucleic acid delivery and expression of foreign DNA in vitro has been very successful. Using the β-lactamase gene, Wong et al. (Gene, 10:87-94, 1980) demonstrated the feasibility of liposome-mediated delivery and expression of foreign DNA in cultured chick embryo, HeLa, and hepatoma cells. Nicolau et al. (supra 1987) accomplished successful liposome-mediated gene transfer in rats after intravenous injection. Also included are various commercial approaches involving “lipofection” technology.

In certain embodiments of the invention, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al, J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, Adv. Drug Delivery Rev., 12:159-167, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, supra 1987) and transferring (Wagner et al., Proc. Nat'l Acad. Sci. USA 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Nat'l Acad. Sci. USA 91:4086-4090, 1994) and epidermal growth factor (EGF) has also been used to deliver genes to squamous carcinoma cells (Myers, EPO 0 273 085).

In other embodiments, the delivery vehicle may comprise a ligand and a liposome. For example, Nicolau et al. (supra 1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a cell type such as muscle, fat, prostate, epithelial or tumor cells, by any number of receptor-ligand systems with or without liposomes. For example, the human prostate-specific antigen (Watt et al., Proc. Nat'l Acad. Sci. USA, 83(2): 3166-3170, 1986) may be used as the receptor for mediated delivery of a nucleic acid in prostate tissue.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (Proc. Nat'l Acad. Sci. USA, 81:7529-7533, 1984) successfully injected polyomavirus DNA in the form of CaPO₄ precipitates into liver and spleen of adult and newborn mice demonstrating active viral replication and acute infection. Benvenisty and Neshif (Proc. Nat'l Acad. Sci. USA, 83:9551-9555, 1986) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., supra 1990). The microprojectiles used have consisted of biologically inert substances such as tungsten or gold beads.

Cells

In still other embodiments, the bioactive agents may be cells. In various embodiments, cells may be associated with the surface of the tissue graft or may be injected or infused into the graft tissue. Exemplary cell types include, for example, cells derived from a variety of tissues such as lung, liver, kidney, thymus, thyroid, heart, brain, pancreas (including acinar and islet cells), mesenchymal cells (including bone, cartilage, ligament, tendon, etc.), especially smooth or skeletal muscle cells, myocytes (muscle stem cells), pluripotent stem cells, progenitor cells, bone marrow stem cells, muscle derived stem cells, blood cells, stem cells from the blood, fat-derived stem cells, mesenchymal stem cells, fibroblasts, chondrocytes, adipocytes, fibromyoblasts, and ectodermal cells, including ductile and skin cells, hepatocytes, Islet cells, cells present in the intestine, and other parenchymal cells, osteoblasts and other cells forming bone or cartilage. In some cases it may also be desirable to include nerve cells. Cells can be unmodified or genetically engineered to produce one or more desired polypeptides, including polypeptides useful as bioactive agents as described above. Methods for genetically engineering cells with retroviral vectors, polyethylene glycol, or other methods known to those skilled in the art can be used.

In exemplary embodiments, cells useful as bioactive agents are autologous to the subject from which the tissue graft was obtained and/or the subject to which the activated tissue graft will be implanted. Alternatively, cells from close relatives or other donors of the same species may be used with appropriate immunosuppression. Immunologically inert cells, such as embryonic or fetal cells, stem cells, and cells genetically engineered to avoid the need for immunosuppression can also be used. Methods and drugs for immunosuppression are known to those skilled in the art of transplantation.

In one embodiment, cells to be used as a bioactive agent may be obtained by biopsy and may optionally be expanded in culture before application to the tissue graft (such as, for example, muscle tissue or fat tissue). Cells can be easily obtained through a biopsy anywhere in the body, for example, skeletal muscle biopsies can be obtained easily from the arm, forearm, or lower extremities; smooth muscle can be obtained from the area adjacent to the subcutaneous tissue throughout the body; and fat tissue can be obtained from almost any subcutaneous region. To obtain either muscle or fat, the area to be biopsied can be locally anesthetized with a small amount of lidocaine injected subcutaneously. Alternatively, a small patch of lidocaine jelly can be applied over the area to be biopsied and left in place for a period of 5 to 20 minutes, prior to obtaining biopsy specimen. The biopsy can be effortlessly obtained with the use of a biopsy needle, a rapid action needle which makes the procedure extremely simple and almost painless. With the addition of the anesthetic agent, the procedure would be entirely painless. This small biopsy core of either skeletal, smooth muscle, or fat can then be transferred to media consisting of phosphate buffered saline. The biopsy specimen is then transferred to the lab where the muscle or fat can be grown utilizing the explant technique, wherein the muscle or fat is divided into very small pieces which are adhered to a culture plate, and serum containing media is added. Alternatively, the muscle or fat biopsy can be enzymatically digested with agents such as trypsin and the cells dispersed in a culture plate with any of the routinely used medias. After cell expansion within the culture plate, the cells can be easily passaged utilizing the usual technique until an adequate number of cells is achieved.

In still other embodiments, the bioactive agents may be cells which naturally produce, or have been engineered to produce, a gene product of interest. Gene products of interest may include, for example, a polypeptide useful as a bioactive agent or a polypeptide which enhances graft stability, maintenance, transformation, etc. once implanted into a subject in need thereof. In certain embodiments, a gene product of interest may be useful to stimulate differentiation of the cell expressing the gene product into a desired cell type. In other embodiments, a gene product of interest may alternatively or additionally stimulate surrounding cells of the tissue graft (such as, for example, muscle tissue or fat tissue) to differentiate into cells of a desired type. In yet another embodiment, a gene product of interest may alternatively or additionally stimulate attraction or ingrowth of pluripotent stem cells and/or progenitor cells to migrate into the tissue graft. After migration into the graft, the same or a different gene product, and/or another bioactive agent, may be used to stimulate differentiation of the stem cells and/or progenitor cells into a desired cell type.

In one embodiment, cells useful as bioactive agents may be expressing one or more gene product used to regulate the growth and/or activity of naturally occurring cells of the host into which the activated tissue graft has been implanted. For example, tumor suppressor gene products may be used to regulate proliferation of the host cells. Regulated expression of tumor suppressor gene products are particularly useful for a variety of applications. For example, one may want the host cells to undergo a rapid proliferation phase followed by a production phase where cellular energies are devoted to protein production, or a rapid proliferation phase in vitro followed by regulated growth in vivo (see, for example, U.S. Pat. No. 6,274,341). Tumor suppressor gene products, as used herein, may be intracellular proteins that block the cell cycle at a cell cycle checkpoint by interaction with cyclins, Cdks or cyclin-Cdk complexes, or by induction of proteins that do so. Thus, these tumor suppressor gene products inhibit the cyclin-dependent progression of the cell cycle. Particularly preferred tumor suppressor gene products act on the G1-S transition of the cell cycle. Any tumor suppressor gene product which performs this function, whether known or yet to be discovered, may be utilized. Examples of tumor suppressor genes include p21, p27, p53 (and particularly, the p53175P mutant allele), p57, p15, p16, p18, p19, p73, GADD45 and APC1.

In other embodiments, the bioactive agents may be cells that express survival factors. Survival factors are intracellular proteins that prevent apoptosis such as bcl-2, bcl-x_{L}, E1B-19K, mc1-1, cimA, ab1, p35, bag-1, A20, LMP-1, Tax, Ras, Rel and NF-κB-like factors. Additionally, all known survival factors, as well as survival factors yet to be discovered, are useful in the methods and compositions disclosed herein. In yet another embodiment, the tumor suppressor gene(s) is expressed concomitantly with a factor that stabilizes the tumor suppressor gene product in the cell. Examples of stabilizing factors are members of the CAAT enhancer binding protein family. For example, p21 protein activity is stabilized when coexpressed with C/EBP-alpha. Additionally, C/EBP-alpha specifically induces transcription of the endogenous p21 gene. Thus, C/EBP-alpha functions as both a stabilizing factor and as a specific inducer of p21.

In still other embodiments, the bioactive agents may be cells that express a gene product that activates cell proliferation. For example, a protein that activates cell proliferation is Mek1, a central protein kinase in the conserved mammalian Ras-MAP signal transduction pathway responding to growth-promoting signals such as cytokines. Other genetic determinants exerting positive control of mammalian cell cycle that can be used as a protein that activates cell proliferation are cyclins (e.g., cyclin E), Ras, Raf, the MAP kinase family (e.g., MAP, Erk, Sap) E2F, Src, Jak, Jun, Fos, pRB, Mek2, EGF, TGF, PDGF, and a polynucleotide that is antisense to a tumor suppressor gene (e.g., p27 antisense expression has been shown to stimulate proliferation of quiescent fibroblasts and enable growth in serum-free medium (Rivard et al., 1996, J. Biol. Chem. 271: 18337-18341) and nedd5 which is known as positive growth controlling gene (Kinoshita et al, 1997, Genes Dev. 11: 1535-1547).

In certain embodiments, the bioactive agents may be cells that express a transcription factor, such as, for example, RUNX and/or osteogenics. In various embodiments, the cells may either naturally express a transcription factor of interest or may be recombinantly engineered to express a transcription factor of interest.

In yet other embodiments, the bioactive agents may be cells that contain genes whose expression can be regulated by external factors. For example, an antibiotic-regulated gene expression in eukaryotic cells based on the repressor of a streptogramin resistance operon of S. coelicolor (a Pip) has been described in U.S. Pat. No. 6,287,813. Briefly, a Pip protein (PIT4), or chimeric Pip proteins (PIT and PIT2) fused to a eukaryotic transactivator can be used to control expression of a synthetic eukaryotic promoter (P_{PIR}) containing the Pₚₜᵣ-binding site (in other words, a P_{abr}-linked gene). Genes placed under the control of this PIT/P_{PIR} system are responsive to clinically approved therapeutic compounds belonging to the streptogramin group (pristinamycin, virginiamycin and Synercid) in a variety of mammalian cell lines (CHO-K1, BHK-21 and HeLa). The well-established tetracycline-based system used in conjunction with CHO cells engineered to provide both streptogramin and tetracycline regulation may also be used.

Exemplary Uses

The methods and compositions disclosed herein may be used for the treatment of lesions in a wide variety of tissues types. In exemplary embodiments, a lesion is identified in a patient which is suitable for treatment by the methods and compositions disclosed herein. Tissue (such as, for example, muscle tissue or fat tissue) is isolated from a subject (optionally from the subject to be treated) and contacted with one or more bioactive agents to induce at least a portion of the cells to differentiate into cells of a desired cell type. In exemplary embodiments, at least a portion of the cells in the tissue are induced to differentiate into cells typically found in the tissue(s) containing the lesion in the subject to be treated. For example, if a patient is suffering from a bone fracture, then the tissue may be treated so as to induce at least a portion of the cells in the tissue to differentiate into bone cells. Bioactive agents suitable for differentiation of cells, such as muscle or fat cells, into cells of a variety of desired cell types are discussed further below.

In various embodiments, at least a portion of the cells in a tissue graft may be induced to differentiate into cells of one or more of the following cell or tissue types: mesodermal tissues, such as mature adipose tissue, bone, cartilage, spinal cord, pancreas, skin, gut, bowel, blood vessels, bladder, joint cartilage, intervertebral disc (nucleus pulposus and anulus fibrosus), ligament, tendon, meniscus, various tissues of the heart (e.g., pericardium, epicardium, epimyocardium, myocardium, pericardium, valve tissue, etc.), dermal connective tissue, hemangial tissues (e.g., corpuscles, endocardium, vascular epithelium, etc.), muscle tissues (including skeletal muscles, cardiac muscles, smooth muscles, etc.), nerve cells, brain cells, urogenital tissues (e.g., kidney, pronephros, meta- and meso-nephric ducts, metanephric diverticulum, ureters, renal pelvis, collecting tubules, epithelium of the female reproductive structures (particularly the oviducts, uterus, and vagina)), splanchnic tissues, liver, pleural and peritoneal tissues, viscera, mesodermal glandular tissues (e.g., adrenal cortex tissues), stromal tissues (e.g., bone marrow), etc. In certain embodiments, it may be desirable to stimulate cells in the tissue to differentiate into a precursor cell (e.g., a preadipocyte, a premyocyte, a preosteocyte, etc.). In other embodiments, different populations of cells in a tissue graft (such as, for example, muscle tissue or fat tissue) may be induced to differentiate into two or more desired cell types. Such embodiments will allow the formation of complex grafts having various cell types or tissue types in the same activated tissue graft.

Various types of cells in a tissue graft can be transduced, activated and/or stimulated to differentiate. For example, in a muscle tissue graft or a fat tissue graft, not only muscle or fat cells may differentiate, but other types of cells that are naturally hosted in such tissues may also differentiate, including, for example, blood cells, nerve cells, fibroblasts, endothelial cells, satellite cells, cells of the immune system, adult stem cells, progenitor cells, etc.

In certain embodiments, after contacting the tissue with one or more bioactive agents, it may be desirable to assay the cells in the tissue to monitor or evaluate the differentiation process. One measurement of differentiation per se is telomere length and/or telomerase activity. Undifferentiated stem cells having longer telomeres than differentiated cells and therefore should also have higher levels of; thus the cells can be assayed for the level of telomerase activity. Alternatively, RNA or proteins can be extracted from the cells and assayed (via Northern hybridization, rtPCR, Western blot analysis, etc.) for the presence of markers indicative of the desired phenotype. Additionally, the cells can be assayed immunohistochemically or stained, using tissue-specific stains. Thus, for example, to assess adipogenic differentiation, the cells can be stained with fat-specific stains (e.g., oil red 0, safarin red, sudan black, etc.) or probed to assess the presence of adipose-related factors (e.g., type IV collagen, PPAR-y, adipsin, lipoprotein lipase, etc.). Similarly, ostogenesis can be assessed by staining the cells with bone-specific stains (e.g., alkaline phosphatase, von Kossa, etc.) or probed for the presence of bone-specific markers (e.g., osteocalcin, osteonectin, osteopontin, type I collagen, bone morphogenic proteins, cbfa, etc.). Myogensis can be assessed by identifying classical morphologic changes (e.g., polynucleated cells, syncitia formation, etc.), or assessed biochemically for the presence of muscle-specific factors (e.g., myo D, myosin heavy chain, NCAM, etc.). Chondrogenesis can be determined by staining the cells using cartilage-specific stains (e.g., alcian blue) or probing the cells for the expression/production of cartilage-specific molecules (e.g., sulfated glycosaminoglycans and proteoglycans (e.g., keratin, chondroitin, etc.) in the medium, type II collagen, etc.). Other methods of assessing developmental phenotype are known in the art, and any of them is appropriate. For example, the cells can be sorted by size and granularity. Also, the cells can be used to generate monoclonal antibodies, which can then be employed to assess whether they preferentially bind to a given cell type. Correlation of antigenicity can confirm that a cell has differentiated along a given developmental pathway.

In an exemplary embodiment, cells are monitored for osteogenic differentiation. The markers of osteogenic differentiation include the expression of messenger RNAs for type I collagen, collagenase (MMP-1), alkaline phosphatase, osteonectin, and TGF-β. These markers are highly characteristic of the osteogenic phenotype. The decreasing expression of osteonectin is also consistent with osteo-differentiation.

In various embodiments, the methods and compositions as described herein may be used to treat a variety of lesions or wounds in a subject, such as, for example, promotion of wound closure including both external (e.g., surface) and internal wounds. Exemplary wound types that may benefit by treatment with the inventive methods and compositions include, but are not limited to, abrasions, avulsions, blowing wounds, burn wounds, contusions, gunshot wounds, incised wounds, open wounds, penetrating wounds, perforating wounds, puncture wounds, seton wounds, stab wounds, surgical wounds, subcutaneous wounds, or tangential wounds. In an exemplary embodiment, wounds, lesions, or injuries of the bone or cartilage, including fractures may be treated. The methods need not achieve complete healing or closure of the wound; it is sufficient for the method to promote any degree of wound closure. In this respect, the method can be employed alone or as an adjunct to other methods for healing wounded tissue.

In various embodiments, one or more bioactive agents may be used to induce at least a portion of the cells in a tissue graft (such as, for example, muscle tissue or fat tissue) to differentiate into cells of a desired cell or tissue type. Appropriate bioactive agents, or combinations of bioactive agents, suitable to induce tissue cells to differentiate into a desired cell type will be available to the skilled artisan based on the teachings herein. Appropriate bioactive agents may also be determined empirically using the methods and procedures described herein.

In one embodiment, one or more BMP (e.g., BMP-2 to BMP-16), IGF, TGF, LMP (e.g., LMP-1, LMP-2, LMP-3) and/or VEGF genes or proteins may be used to induce differentiation of cells, such as, for example, muscle tissue cells or fat tissue cells, into bone cells. The osteoinductive activity of LMP proteins are described, for example, in Minamide et al., J. Bone Joint Surg. Am. 85-A: 1030-9 (2003), Kim et al., Spine 28: 219-26 (2003), Liu et al., J. Bone Miner. Res. 17: 406-14 (2002), Viggeswarapu et al., J. Bone Joint Surg. Am. 83-A: 364-76 (2001), O'Reilly et al., Immunol. Rev. 157: 195-216 (1997), and Boden et al., Endocrinology 139: 5125-34. The role of VEGF in bone formation and/or repair is described, for example, in Tarkka et al., J. Gene Med. 5: 560-6 (2003), Furumatsu et al., J. Biochem (Tokyo) 133: 633-9 (2003), Kumta et al., Lif. Sci. 73: 1427-36 (2003), and Khodaparast et al., Plast. Reconstr. Surg. 112: 171-6 (2003). The role of IGF in bone formation and/or repair is described, for example, in Okazaki et al., J. Orthop. Res. 21: 511-20 (2003). In an exemplary embodiment, a BMP-2 and/or BMP-7 polypeptide is used as a bioactive agent to induce differentiation of muscle or fat tissue cells into bone cells. In another exemplary embodiment, a polypeptide having SEQ ID NO: 2 and/or SEQ ID NO: 4 may be used as a bioactive agent to induce differentiation of muscle or fat tissue cells into bone cells.

In another embodiment, one or more SOX (e.g., SOX-5, SOX-6, SOX-9), BMP (e.g., BMP-2 to BMP-16), IGF, PDGF, EGF, CDMP, 1HH, PTHrP, cartilage growth factor (CGF) and/or TGF genes or proteins may be used to induce formation of cartilage cells from muscle or fat tissue cells. The role of IHH in cartilage repair and/or formation is described, for example, in Webster et al., Avian Pathol. 32: 69-80 (2003), Semevolos et al. J. Orthop. Res. 20: 1290-7 (2002), de Crombrugghe et al., Curr. Opin. Cell Biol. 13: 721-7 (2001). The role of PTHrP in cartilage repair and/or formation is described, for example, in Webster et al., Avian Pathol. 32: 69-80 (2003), Okazaki et al., J. Orthop. Res. 21: 511-20 (2003), Semevolos et al. J. Orthop. Res. 20: 1290-7 (2002), Rabie et al., J. Dent. Res. 82: 627-31 (2003), de Crombrugghe et al., Curr. Opin. Cell Biol. 13: 721-7 (2001). The role of IGF in cartilage repair and/or formation is described, for example, in Okazaki et al., J. Orthop. Res. 21: 511-20 (2003). The role of SOX in cartilage repair and/or formation is described, for example, in Fernandez et al., Mol. Endocrinol. 17: 1332-43 (2003), Akiyama et al., Genes Dev. 16: 2813-28 (2002), Yang and Karsenty, Trends Mol. Med. 8: 340-5 (2002), Uusitalo et al., J. Bone Miner. Res. 16: 1837-45 (2001), Reppe et al., J. Bone Miner. Res. 15: 2402-12 (2000), DeLise et al., Osteoarthritis 8: 309-34 (2000), and Lefebvre et al., Mol. Cell Biol. 17: 2336-46 (1997).

In yet another embodiment, nerve growth factor (NGF) genes or proteins may be used to induce nerve cell differentiation.

In still another embodiment, endothelial cell differentiation may be induced in the presence of one or more of the following genes or proteins: platelet-derived growth factor (PDGF), vascular endothelial growth factor (VEGF), insulin-like growth factor (IGF-1), keratinocyte growth factor (KGF), endothelial derived growth supplement (EDGF), epidermal growth factor (EGF), and basic fibroblast growth factor (FGF).

In an exemplary embodiment, one or more bioactive agents are used to induce formation of bone cells from muscle or fat tissue cells. It is well established that certain bioactive molecules can induce formation of bone or connective tissue. Members of the TGF-beta superfamily appear to play a central role in mesenchymal differentation, including cartilage and bone formation. TGF-beta enhances bone cell proliferation. The TGF-beta superfamily includes the bone morphogenic proteins, including BMP-2-16 and Insulin-like growth factor (IGF). These. can be further divided into three distinct subfamilies: BMP-2, BMP-3, and BMP-7. The different isoforms have different activities in bone morphogenesis and repair. They are closely related to factors which are involved in a variety of developmental processes during embryogenesis. For example, any of BMP 2-7 can be used to induce bone formation and differentiation. IGF has been shown to increase bone formation, promoting fracture healing and inducing bone growth around implants, in conjunction with TGF-beta and BMPs. Other osteoinductive factors such as osteogenin (BMP-3), a skeletal growth factor (SGF), and osteoblast-derived (BDGFs) have also been recently discovered. Other factors shown to act on cells forming bone, cartilage or other connective tissue include retinoids, fibroblast growth factors (FGFs), growth hormone (GH), and transferrin. Proteins specific for cartilage repair include cartilage growth factor (CGF) and TGF-beta. The local microenvironment also affects differentiation and development of cells. In an exemplary embodiment, BMP-2 and/or BMP-7 may be used to induce muscle or fat tissue cells to differentiate into bone cells.

The activated tissue grafts described herein may be used as a pure delivery system that releases one or more drugs (such as, for example, hormones, growth factors, antibodies, etc.) systemically into the body or locally at a specific site. For example, an activated tissue graft may be implanted into a lesion to release a drug to a specific site in need, a graft may be wrapped around a lesion (e.g., a bone lesion, fracture, etc.) to release a drug to the lesion, or the graft may be implanted under the skin to release drugs into the blood stream, etc. An activated tissue graft useful as a drug delivery system need not transform into another tissue type but rather serves as a drug delivery system that may slowly degrade over time.

An activated tissue graft may partially or fully transform into a desired tissue through differentiation of cells in the graft. Such differentiation may take place either outside the body prior to implantation, inside the body after implantation, or a combination thereof. Alternatively, the graft may slowly degrade over time and be replaced through ingrowth of the surrounding defect tissue. Ingrowth of surrounding tissue and degradation of the graft may be regulated by bioactive agents that are released by the graft. In this case, cells in the graft may not differentiate.

In one embodiment, the activated tissue graft may be mechanically stimulated (e.g., compression or stretching of the tissue) to enhance and accelerate the transformation into a desired tissue. Such mechanical stimulation may occur ex vivo or in situ.

In another embodiment, an activated tissue graft, such as, for example, an activated muscle or fat graft, may be used in conjunction with a variety of biomaterials, including, for example, hydroxyapatite, tricalcium phosphate, calcium sulfate, polylactic acid, collagen, etc. for the repair of a lesion, such as, for example, a bone or cartilage lesion in a subject. For example, a piece or thin sheet of tissue can be applied to a biomaterial before implantation into a subject. The tissue may be attached to the biomaterial by methods known to the skilled artisan based on the teachings herein, including, for example, physical attachment (e.g., via a screw, clip, pin, etc.), chemical attachment (e.g., via a glue or adhesive), or a biochemical attachment (e.g., via a polypeptide or other biomolecule which promotes adhesion of the tissue to the biomaterial). The biomaterial may facilitate at least a portion of the cells in the tissue to differentiate into cells of a desired type and/or may serve as a scaffold for the tissue. In certain embodiments, the biomaterial may naturally stimulate at least a portion of the cells in the tissue (such as, for example, muscle tissue or fat tissue) to differentiate into cells of a desired type. Alternatively, the biomaterial may be modified to stimulate at least a portion of the cells in the tissue to differentiate into cells of a desired type, for example, by mixing and/or coating the biomaterial with one or more bioactive agents.

In another embodiment, an activated tissue graft, such as, for example, an activated muscle or fat graft, may be used in conjunction with an autograft or an allograft for treatment of a lesion in a subject. In an exemplary embodiment, an activated tissue graft may be used in conjunction with one or more of a bone autograft, bone allograft, bone xenograft, cartilage autograft, cartilage allograft, or cartilage xenograft for treatment of a bone and/or cartilage lesion in a subject. The bone and/or cartilage graft may serve as a scaffold for the activated tissue graft and/or may facilitate stimulation of at least a portion of the cells in the tissue to differentiate into cells of a desired type.

A variety of bone injuries, diseases, disorders and conditions may be treated using the methods and compositions described herein for the formation of bone from tissue, such as muscle or fat tissue. Any disease or disorder that would benefit from improved bone repair or healing processes may treated in accordance with the methods of the invention, including, for example, bone fractures, such as those suffered by healthy individuals or individuals suffering from a disease or disorder such as vitamin D deficiency, osteogenesis imperfecta, or osteoporosis.

In one embodiment, the methods and compositions disclosed herein may be used to treat an otherwise healthy individual who suffers a fracture. Often, clinical bone fracture is treated by casting to alleviate pain and a low natural repair mechanisms to repair the wound. There has been progress in the treatment of fracture in recent times, however, even without considering the various complications that may arise in treating fractured bones, any new procedures to increase bone healing in normal circumstances would represent a great advance.

In another embodiment, an individual suffering from osteogenesis imperfecta (OI) may be treated in accordance with the methods and compositions described herein. OI encompasses various inherited connective tissue diseases that involve bone and soft connective tissue fragility in humans. About one child per 5,000-14,000 born is affected with OI and the disease is associated with significant morbidity throughout life. A certain number of deaths also occur, resulting from the high propensity for bone fracture and the deformation of abnormal bone after fracture repair (OI types II-IV). The relevant issue here is quality of life; clearly, the lives of affected individuals would be improved by the development of new therapies designed to stimulate and strengthen the fracture repair process.

In yet another example, the methods and compositions described herein may be used to treat an individual suffering from osteoporosis. The term osteoporosis refers to a heterogeneous group of disorders characterized by decreased bone mass and fractures. An estimated 20-25 million people are at increased risk for fracture because of site-specific bone loss. Risk factors for osteoporosis include increasing age, gender (more females), low bone mass, early menopause, race (Caucasians), low calcium intake, reduced physical activity, genetic factors, environmental factors (including cigarette smoking and abuse of alcohol or caffeine), and deficiencies in neuromuscular control that create a propensity to fall.

More than a million fractures in the USA each year can be attributed to osteoporosis, and in 1986 alone the treatment of osteoporosis cost an estimated 7-10 billion health care dollars. Demographic trends (i.e., the gradually increasing age of the US population) suggest that these costs may increase to $62 billion by the year 2020. Clearly, osteoporosis is a significant health care problem.

Clinically, osteoporosis is segregated into type I and type II. Type I osteoporosis occurs predominantly in middle aged women and is associated with estrogen loss at the menopause, while osteoporosis type II is associated with advancing age. Much of the morbidity and mortality associated with osteoporosis results from immobilization of elderly patients following fracture.

Current therapies for osteoporosis patients focus on fracture prevention, not fracture repair. This remains an important consideration because of the literature, which clearly states that significant morbidity and mortality are associated with prolonged bed rest in the elderly, particularly those who have suffered hip fractures. Complications of bed rest include blood clots and pneumonia. These complications are recognized and measures are usually taken to a void them, but these is hardly the best approach to therapy. Thus, the osteoporotic patient population would benefit from new therapies designed to strengthen bone and speed up the fracture repair process, thus getting these people on their feet before the complications arise.

In another embodiment, bone reconstruction and, specifically, the ability to reconstruct defects in bone tissue that result from traumatic injury; as a consequence of cancer or cancer surgery; as a result of a birth defect; or as a result of aging; may be treated in accordance with the methods and compositions disclosed herein. There is a significant need for improved orthopedic implants, and cranial and facial bone are particular targets for this type of reconstructive need. The availability of new implant materials, e.g., titanium, has permitted the repair of relatively large defects. Titanium implants provide excellent temporary stability across bony defects. However, experience has shown that a lack of viable bone bridging the defect can result in exposure of the appliance, infection, structural instability and, ultimately, failure to repair the defect.

Autologous bone grafts are another possibility, but they have several demonstrated disadvantages in that they must be harvested from a donor site such as iliac crest or rib, they usually provide insufficient bone to completely fill the defect, and the bone that does form is sometimes prone to infection and resorption. Partially purified xenogeneic preparations are not practical for clinical use because microgram quantities are purified from kilograms of bovine bone, making large scale commercial production both costly and impractical. Allografts and demineralized bone preparations are therefore often employed.

Microsurgical transfers of free bone grafts with attached soft tissue and blood vessels can close bony defects with an immediate source of blood supply to the graft. However, these techniques are time consuming, have been shown to produce a great deal of morbidity, and can only be used by specially trained individuals. Furthermore, the bone implant is often limited in quantity and is not readily contoured. In the mandible, for example, the majority of patients cannot wear dental appliances using presently accepted techniques (even after continuity is

In another exemplary embodiment, the methods and compositions of the invention are used to repair a lesion in cartilage tissue, such as cartilage tears, arthritis, congenital or trauma induced cartilage defects, etc. or other defects. Cartilaginous tissue refers to tissue which is formed by chondrocytes and which demonstrate the histological and compositional characteristics of cartilage. In one embodiment, bioactive agents which are useful in inducing formation of chondrocytes or cartilage from muscle or fat tissue include the Transforming Growth Factor-Beta (TGF-beta) superfamily of proteins, such as the Bone Morphogenetic Proteins (BMPs) and the Growth and Differentiation Factors (GDFs).

To carry out the methods of treating defects or lesions in a patient according to the methods disclosed herein, a defect to be repaired is first identified. Defects or lesions in a subject may be readily identified visually during open surgery or by minimally invasive surgical techniques. Defects may also be identified using imaging techniques such as, for example, computer aided tomography (CAT scanning), X-ray examination, magnetic resonance imaging (MRI), serum markers, or by any other procedure known in the art.

Once a defect has been identified, prior to, or at the time of repair, the surgeon may elect to surgically modify the defect to enhance the ability of the defect to physically retain the graft being implanted in accordance with the compositions and methods disclosed herein. For example, a lesion or defect having a flat or shallow concave geometry may be altered or shaped to better receive and/or retain the graft.

In certain embodiments, the graft may be secured at defect site in a patient using physical, chemical, and/or biological devices or agents known in the art. For example, a graft may be affixed at a desired location within a patient using plates, screws, pins, sutures, etc. In exemplary embodiments, such fixation devices are made out of biocompatible materials which may optionally be biodegradable and/or bioerodible. In other embodiments, a chemical and/or biological agent may be used to facilitate fixation of the graft at a desired location within a patient. Such chemical and/or biological agents may be used alone or coupled with a physical fixation device. Exemplary chemical and/or biological agents include, for example, glues or adhesives. For example, the defect site may be treated with a compound, such as fibrin glue or transglutaminase, to enhance adhesion of the graft to the defect site. In certain embodiments, the fibrin glue or transglutaminase may be applied to the defect site after the defect site has been rinsed and dried following enzyme treatment. Fibrin glue and transglutaminase promote chemical bonding (cross-linking) of the graft to the defect surface (see e.g., Gibble et al., Transfusion, 30: 741-47 (1990); Ichinose et al., J. Biol. Chem., 265: 13411-14 (1990); “Transglutaminase,” Eds: V. A. Najjar and L. Lorand, Martinus Nijhoff Publishers (Boston, 1984)). Suturing, cauterization or compounds other than fibrin glue or transglutaminase that can promote adhesion of extracellular materials may also be used.

In one embodiment, when implanting the graft into a bone or cartilage lesion, the defect site may be treated prior to implantation of the graft with a proteoglycan-degrading enzyme and/or other materials to improve adhesion of the graft into the defect site of a patient. For example, the surface of the defect may be dried by blotting the area using sterile absorbent tissue, and the defect volume may be filled with a sterile enzyme solution for a period of 2-10 minutes to degrade the proteoglycans present on the surface of the bone or cartilage and locally within approximately 1 to 2μ deep from the surface of the defect. Various enzymes may be used, singly or in combination, in sterile buffered aqueous solutions to degrade the proteoglycans. The pH of the solution may be adjusted to optimize enzyme activity. Enzymes useful to degrade the proteoglycans include chondroitinase ABC, chondroitinase AC, hyaluronidase, pepsin, trypsin, chymotrypsin, papain, pronase, stromelysin and Staph V8 protease (Jurgensen, K. et al., J. Bone Joint Surg. Am., 79: 185-93 (1997); Hunziker, E. B. et al., J. Bone Joint Surg. Br., 80: 144-50 (1998)). The appropriate concentration of a particular enzyme or combination of enzymes will depend on the activity of the enzyme solution and may be determined by one of ordinary skill in the art based on the teachings herein.

In certain embodiments, it may be desirable to administer one or more therapeutic agents to a patient receiving the graft before, concurrently with, or after implantation of the graft into the patient. In various embodiments it the therapeutic agent may be applied to the graft and/or administered to the patient via a traditional route. Appropriate dosages and methods of administration will be apparent to one of skill in the art based on the teachings herein.

Exemplary therapeutic agents include, for example, anti-inflammatory agents, immunosuppressive agents, and/or anti-infective agents (such as for example, antibiotic, antiviral, and/or antifungal compounds, etc.). Exemplary anti-inflammatory drugs include, for example, steroidal (such as, for example, cortisol, aldosterone, prednisone, methylprednisone, triamcinolone, dexamethasone, deoxycorticosterone, and fluorocortisol) and non-steroidal anti-inflammatory drugs (such as, for example, ibuprofen, naproxen, and piroxicam). Exemplary immunosuppressive drugs include, for example, prednisone, azathioprine (Imuran), cyclosporine (Sandimmune, Neoral), rapamycin, antithymocyte globulin, daclizumab, OKT3 and ALG, mycophenolate mofetil (Cellcept) and tacrolimus (Prograf, FK506). Exemplary antibiotics include, for example, sulfa drugs (e.g., sulfanilamide), folic acid analogs (e.g., trimethoprim), beta-lactams (e.g., penacillin, cephalosporins), aminoglycosides (e.g., stretomycin, kanamycin, neomycin, gentamycin), tetracyclines (e.g., chlorotetracycline, oxytetracycline, and doxycycline), macrolides (e.g., erythromycin, azithromycin, and clarithromycin), lincosamides (e.g., clindamycin), streptogramins (e.g., quinupristin and dalfopristin), fluoroquinolones (e.g., ciprofloxacin, levofloxacin, and moxifloxacin), polypeptides (e.g., polymixins), rifampin, mupirocin, cycloserine, aminocyclitol (e.g., spectinomycin), glycopeptides (e.g., vancomycin), and oxazolidinones (e.g., linezolid). Exemplary antiviral agents include, for example, vidarabine, acyclovir, gancyclovir, valganciclovir, nucleoside-analog reverse transcriptase inhibitors (e.g., ZAT, ddI, ddC, D4T, 3TC), non-nucleoside reverse transcriptase inhibitors (e.g., nevirapine, delavirdine), protease inhibitors (e.g., saquinavir, ritonavir, indinavir, nelfinavir), ribavirin, amantadine, rimantadine, relenza, tamiflu, pleconaril, and interferons. Exemplary antifungal drugs include, for example, polyene antifungals (e.g., amphotericin and nystatin), imidazole antifungals (ketoconazole and miconazole), triazole antifungals (e.g., fluconazole and itraconazole), flucytosine, griseofulvin, and terbinafine.

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Treatment of a Critical Size Defect in a Rat Model Using an Activated Muscle Graft

Vector Production. A first generation adenoviruses carrying human BMP-2 cDNA (GenBank Accession No. NM_{—}001200) and the LacZ gene was constructed. First-generation, E1, E3-deleted, serotype 5 recombinant adenoviral vectors containing BMP-2 were constructed using Cre-lox recombination by the system of Hardy et al. (J. Virol., 71(3): 1842-9 (1997)). The resulting vector was designated Ad.BMP-2. For this system, the gene of interest is inserted directionally into an adenoviral shuttle plasmid, pAdlox, containing the 3′inverted terminal repeat of the virus, a viral packaging signal (ψ), a cDNA expression cassette driven by the cytomegalovirus promoter/enhancer, and, finally, a loxP Cre recombinase recognition sequence. Recombinant adenovirus is generated by cotransfection of linearized Adlox shuttle plasmid with ψ5 adenoviral genomic DNA, which has its packaging sequence flanked by loxP sites. The transfection is performed in a cell line called Cre8, which constitutively expresses high levels of Cre recombinase.

The transfected Cre8 cells generate recombinant adenoviral particles following Cre-mediated recombination between the loxP site in the shuttle vector and the 3′loxP site in the ψ5 adenoviral backbone. Propagation of nonrecombined ψ5 virus is selected against via deletion of the packaging signal by the Cre recombinase. Plaques isolated from the cotransfected plates are almost exclusively recombinants. Any contaminating ψ5 can be eliminated by subsequent propagation of the adenovirus in 293 Cre8 cells, or by plaque purification if necessary. To generate recombinant virus for large-scale infections, replication-deficient adenovirus is typically amplified in 293 cells and purified by standard cesium chloride banding techniques. Viral titers were determined by optical density at 260 nm (1 unit=10¹² virions/ml).

Critical sized defect model. 5 mm segmental, critical sized defects were created in the right femora of Sprague-Dawley rats with a dental burr. The bones were stabilized by an external fixator with 1.1 mm Kirschner wires as pins using the method of (Einhorn, T. A., et al. J Bone Joint Surg Am 66(2), 274-9 (1984)).

A small muscle graft approximately of the size of the bone defect was taken from the upper thigh muscle. A dose of 4×10¹⁰ viral particles (4×10⁸ pfu) was then administered by multiple injections into the free muscle graft using a micro-syringe under sterile conditions. The modified muscle tissue was then placed into the defect and the surrounding muscle was then closed around the lesion.

Control animals were treated as it was described above, but without injecting virus vector into the free muscle graft.

Healing was monitored by weekly X-ray. Hematoxylin-eosin (HE) staining was used to monitor undecalcified bone histology (see e.g., An & Martin, Handbook of Histology Methods for Bone and Cartilage, Humana Press, Totowa, N.J., 2003).

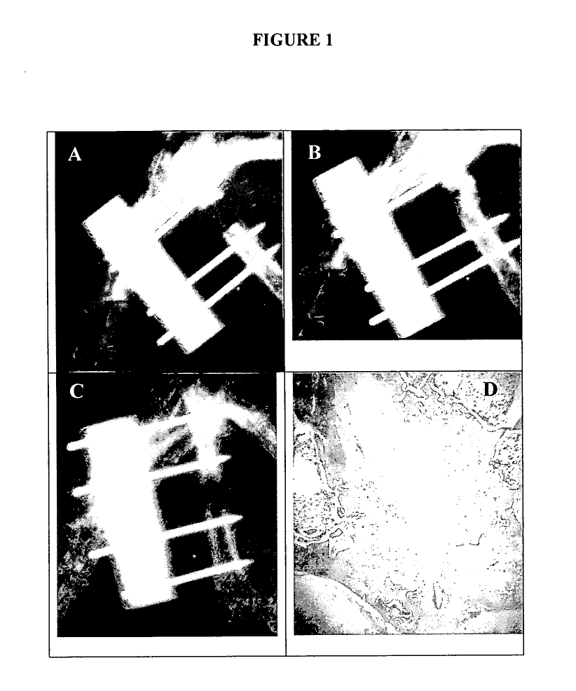

Immediately after surgery the defects between the distal and the proximal femur had the same size in all groups (FIG. 1A). Healed defects were defined as those with bone bridging of at least 75% of the defect site. FIG. 1A shows the defect one day after surgery. After 6 weeks, the defects treated with the BMP-2 gene transduced muscle tissue clearly showed complete bridging (FIG. 1B). Histological evaluation (Haematoxilin/Eosin staining) of the same specimen also showed massive bone formation in the gap area after 6 weeks in the femur treated with the activated muscle graft (FIG. 1D), whereas none of the control rats showed signs of healing after 6 weeks (FIG. 1C).

### Example 2

### Treatment of a Critical Size Defect in a Rat Model Using an Activated Fat Graft

Vector Production. A first generation adenoviruses carrying human BMP-2 cDNA (GenBank Accession No. NM_{—}001200) and the LacZ gene was constructed as described in EXAMPLE 1.

Critical sized defect model. 5 mm segmental, critical sized defects were created in the right femora of Sprague-Dawley rats with a dental burr as described in EXAMPLE 1. The bones were stabilized by an external fixator with 1.1 mm Kirschner wires as pins using the method of (Einhorn, T. A., et al. J Bone Joint Surg Am 66(2), 274-9 (1984)).

A small fat graft approximately of the size of the bone defect was taken from the subcutaneous fat depot. A dose of 4×10¹⁰ viral particles (4×10⁸ pfu) was then administered by multiple injections into the free fat graft using a micro-syringe under sterile conditions. The fat tissue was then cultured for 5 hours in the lab until infection and transduction of fat cells was completed. The modified fat tissue was then placed into the bone defect.

Control animals were treated as it was described above, but without injecting virus vector into the free fat graft.

Healing was monitored by weekly X-ray. Hematoxylin-eosin (HE) staining was used to monitor undecalcified bone histology (see e.g., An & Martin, Handbook of Histology Methods for Bone and Cartilage, Humana Press, Totowa, N.J., 2003).

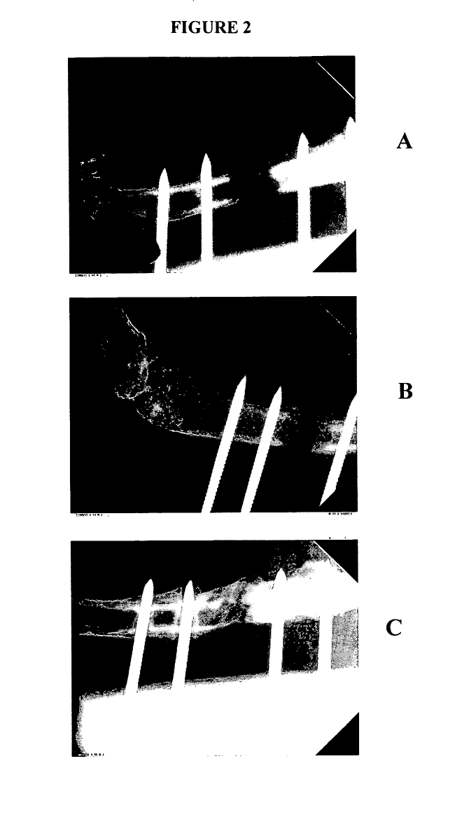

Immediately after surgery the defects between the distal and the proximal femur had the same size in all groups. Healed defects were defined as those with bone bridging of at least 75% of the defect site. FIG. 2A shows an untreated defect 8 weeks after surgery. FIG. 2B shows a defect treated with an unmodified fat graft 8 weeks after surgery. FIG. 2C shows a defect treated with an Ad.BMP-2 actovated fat graft 8 weeks after surgery. After 8 weeks, the defects treated with the BMP-2 gene transduced fat tissue clearly showed complete bridging (FIG. 2C), whereas none of the control rats showed signs of healing after 8 weeks (FIGS. 2A and 2B).

Equivalents

The present disclosure provides among other things methods and compositions for tissue repair. While specific embodiments have been discussed, the above specification is illustrative and not restrictive. Many variations of the methods, compositions, and process disclosed herein will become apparent to those skilled in the art upon review of this specification. The appended claims are not intended to claim all such embodiments and variations, and the full scope of the invention should be determined by reference to the claims, along with their full scope of equivalents, and the specification, along with such variations.

Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control. Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: U.S. Pat. No. 6,503,227 B2; WO 02/10348 A2; U.S. 2002/0007223 A1; WO 02/10348 A2; WO 02/22184 A2; U.S. Pat. No. 6,486,133 B1; U.S. 2002/0076816; U.S. Pat. No. 6,391,297 B1; WO 01/82773 A2; U.S. 2002/0127711 A1; WO 02/060315 A2; WO 01/82973 A2; WO 02/067867 A2; U.S. 2002/0098168 A1; WO 01/66130 A1; WO 01/08714 A1; WO 02/067978 A1; U.S. 2002/0076400 A1; U.S. 2002/0193338 A1; U.S. Pat. No. 5,763,416; U.S. Pat. No. 6,398,816 B1; Gulbins H et al, Heart Surg Forum 2002, 5(4):E28-34; O brien K et al., J Cell Biochem Suppl 2002, 38:80-7; Zammit P et al., Differentiation 2001 October, 68)4-5):193-204; Rende M et al., Int J Dev Neurosci 2000 December, 18(8):869-85; Musgrave D S et al., Bone 2001 May; 28(5):499-506; Musgrave D S et al., Clin Orthop 2000 September, (378):290-305; Bosch P et al., Cell Transplant 2000 July-August, 9(4):463-70; Bosch P et al., J Orthop Res 2000 November, 18(6):933-44; Evans C H et al., Clin Orthop 2000 October, (379 Suppl):S214-9; Goldstein S A et al., Clin Orthop 1998 October, (355 Suppl):S154-2; Baltzer A W et al., Knee Surg Sport Traumatol Arthrosc 1999; 7(3): 197-202; Baltzer A W et al., Gene Ther 2000 May, 7(9):734-9; Musgrave D S et al., Bone 1999 June; 24(6):541-7; Ripamonti U et al., Curr Pharm Biotechnol 2000 July, 1(1):47-55; Levy R J et al., Adv Drug Deliv Rev 1998 Aug. 3, 33(1-2):53-69; Bonadio J et al., Nat Med 1990 July, 5(7):753-9; Goldstein S A, Clin Orthop 2000 October, (379 Suppl):S113-9; Bonadio J., Adv Drug Deliv Rev 2000 Nov. 15, 44(2-3):185-94; Ripamont U, et al., Growth Factors 2000, 17(4):269-85; Michiko R. Wada et al., Development and Disease; Adachi N, et al. J Rheumatol. 29(9): 1920-30 (2002); Lee C W, et al. J Pediatr Orthop. 22(5): 565-72 (2002); Cao B, et al. Nat Cell Biol. 5(7): 640-6 (2003); and Jon S. Odorico et al., Stem Cells 2001,19:193-204.

1. A method for producing an activated tissue graft comprising contacting tissue with one or more bioactive agents ex vivo, wherein the bioactive agents stimulate at least a portion of the cells in the tissue to differentiate into cells of a desired type, thereby producing an activated tissue graft.

2. The method of claim 1, wherein the tissue is muscle tissue.

3. The method of claim 2, wherein the muscle tissue is skeletal muscle tissue.

4. The method of claim 1, wherein the tissue is fat tissue.

5. The method of claim 4, wherein the fat tissue is subcutaneous fat depot.

6. The method of claim 1, wherein the bioactive agents may be one or more of the following: polypeptides, nucleic acids, hormones, cells, drugs and small molecules.

7. The method of claim 6, wherein the bioactive agents are one or more polypeptides.

8. The method of claim 7, wherein the bioactive agents are one or more of the following: growth factor or signaling molecule.

9. The method of claim 6, wherein the bioactive agents are one or more cells.

10. The method of claim 9, wherein the cells are stem cells.

11. The method of claim 9, wherein the cells are transformed with at least one nucleic acid encoding a bioactive polypeptide.

12. The method of claim 11, wherein the bioactive polypeptide is a signaling molecule.

13. The method of claim 6, wherein the bioactive agents are one or more nucleic acids.

14. The method of claim 13, wherein the nucleic acids encode a bioactive polypeptide.

15. The method of claim 13, wherein the nucleic acids are contained on an expression vector.

16. The method of claim 15, wherein the expression vector is a viral vector.

17. The method of claim 16, wherein the viral vector is an adenoviral vector.

18. The method of claim 13, wherein the nucleic acids are formulated with one or more agents that facilitate uptake of the nucleic acids by at least a portion of the cells in the tissue.

19. The method of claim 18, wherein the nucleic acids are formulated with one or more of the following: nanocaps, colloidal gold, nanoparticulate synthetic polymers, or liposomes

20. The method of claim 1, wherein contacting the tissue with one or more bioactive agents comprises one or more of coating, soaking, or injecting the tissue with the bioactive agents.

21. The method of claim 1, wherein the bioactive agents are formulated in a time release formulation.

22. The method of claim 1, wherein the tissue has a three dimensional shape suitable for repair of a lesion in a subject.

23. The method of claim 22, wherein the tissue is removed from the subject in a three dimensional shape suitable for repair of a lesion in the same or a different subject.

24. The method of claim 22, wherein the tissue is formed into a three dimensional shape suitable for repair of a lesion in a subject after removal from the same or a different subject.

25. The method of claim 1, wherein the tissue is incubated ex vivo for at least one hour after being contacted with the one or more bioactive agents.

26. The method of claim 25, wherein the tissue is incubated ex vivo for at least 24 hours after being contacted with the one or more bioactive agents.

27. The method of claim 1, wherein contacting the tissue with one or more bioactive agents occurs at least twice.

28. The method of claim 1, wherein the tissue begins to at least partially transform into a desired tissue type prior to implantation into a subject.

29. The method of claim 1, wherein the activated tissue graft is autologous, allogenic, or xenogeneic to a subject that receives the activated tissue graft.

30. The method of claim 1, wherein the cells of a desired type are cells of one or more of the following tissues: bone, cartilage, ligament, tendon, heart, liver, kidney, brain, skin, cartilage, bladder, lung, thymus, thyroid, spinal cord, pancreas, skin, gut, bowel, blood vessels, bladder, joint cartilage, intervertebral disc, ligament, tendon, meniscus, or pancreas.

31. The method of claim 1, wherein the bioactive agents are osteoinductive molecules.

32. The method of claim 31, wherein at least a portion of the cells in the tissue graft differentiate into bone cells.

33. The method of claim 30, wherein at least a portion of the cells in the tissue graft differentiate into cartilage cells.

34. The method of claim 31, wherein the osteoinductive molecules are one or more of the following: BMP-1, BMP-2, BMP-3, BMP-4, BMP-5, BMP-6, BMP-7, BMP-8, BMP-9, BMP-10, BMP-1, BMP-12, BMP-13, BMP-14, BMP-15 and BMP-16.

35. The method of claim 34, wherein the osteoinductive molecules are one or more of BMP-2 and BMP-7.

36. The method of claim 1, wherein the method further comprises contacting the tissue with a therapeutic agent.

37. The method of claim 36, wherein the therapeutic agent is one or more of the following: an antibiotic, an antiviral, an antifungal, an immunosuppressive and an antiinflammatory agent.

38. A method for producing an activated tissue graft comprising contacting tissue ex vivo with at least one nucleic acid encoding at least one bioactive polypeptide thereby introducing said nucleic acid(s) into at least a portion of the cells in said tissue, wherein the bioactive polypeptides are expressed in said cells, and wherein the bioactive polypeptides stimulate at least a portion of the cells in said tissue to differentiate into cells of a desired type, thereby forming an activated tissue graft.

39. The method of claim 38, wherein said tissue is muscle tissue.

40. The method of claim 39, wherein said muscle tissue is skeletal muscle tissue.

41. The method of claim 38, wherein said tissue is fat tissue.

42. The method of claim 41, wherein said fat tissue is subcutaneous fat depot.

43. The method of claim 38, wherein the cells of a desired type are cells of one or more of the following tissues: bone, cartilage, ligament, tendon, heart, liver, kidney, brain, skin, cartilage, bladder, lung, thymus, thyroid, spinal cord, pancreas, skin, gut, bowel, blood vessels, bladder, joint cartilage, intervertebral disc, ligament, tendon, meniscus, or pancreas.

44. The method of claim 38, wherein the bioactive polypeptides are osteoinductive proteins.

45. The method of claim 44, wherein at least a portion of the cells in the tissue graft differentiate into bone cells.

46. The method of claim 44, wherein the osteoinductive proteins are one or more of the following: BMP-1, BMP-2, BMP-3, BMP-4, BMP-5, BMP-6, BMP-7, BMP-8, BMP-9, BMP-10, BMP-11, BMP-12, BMP-13, BMP-14, BMP-15 and BMP-16.

47. The method of claim 46, wherein the osteoinductive molecules are one or more of BMP-2 and BMP-7.

48. A composition comprising tissue and one or more bioactive agents, wherein the bioactive agents stimulate at least a portion of the cells in the tissue to differentiate into cells of a desired type.

49. The composition of claim 48, wherein the tissue is muscle tissue.

50. The composition of claim 49, wherein the muscle tissue is skeletal muscle tissue.

51. The composition of claim 48, wherein the tissue is fat tissue.

52. The composition of claim 51, wherein the fat tissue is subcutaneous fat depot.

53. The composition of claim 48, wherein the bioactive agents may be one or more of the following: polypeptides, nucleic acids, hormones, cells, drugs and small molecules.

54. The composition of claim 53, wherein bioactive agents are one or more of the following: at least one osteoinductive polypeptide and at least one nucleic acid encoding an osteoinductive polypeptide.

55. The composition of claim 54, wherein at least a portion of the cells in the tissue graft differentiate into bone cells.

56. The composition of claim 54, wherein the osteoinductive polypeptides are one or more of the following: BMP-1, BMP-2, BMP-3, BMP-4, BMP-5, BMP-6, BMP-7, BMP-8, BMP-9, BMP-10, BMP-1, BMP-12, BMP-13, BMP-14, BMP-15 and BMP-16.

57. The composition of claim 56, wherein the osteoinductive polypeptides are one or more of BMP-2 and BMP-7.

58. An activated muscle graft comprising muscle tissue which has been exposed to one or more bioactive agents, wherein the bioactive agents stimulate at least a portion of the cells in the muscle tissue to differentiate into cells of a desired type.

59. An activated fat graft comprising fat tissue which has been exposed to one or more bioactive agents, wherein the bioactive agents stimulate at least a portion of the cells in the fat tissue to differentiate into cells of a desired type.

60. A method for treating a lesion in a subject comprising:
1) contacting tissue with one or more bioactive agents ex vivo, wherein the bioactive agents stimulate at least a portion of the cells in the tissue to differentiate into cells of a desired type; and
2) implanting the tissue into a lesion of the subject.

61. The method of claim 60, wherein at least a portion of the cells in the tissue differentiate into a type of cell found in the tissue comprising the lesion in the subject.

62. The method of claim 60, wherein the tissue is muscle tissue.

63. The method of claim 62, wherein the muscle tissue is skeletal muscle tissue.

64. The method of claim 60, wherein the tissue is fat tissue.

65. The method of claim 64, wherein the fat tissue is subcutaneous fat depot.

66. The method of claim 60, wherein the tissue is removed from the subject in an appropriate size and shape for implantation into the lesion.

67. The method of claim 60, wherein the tissue is configured to an appropriate size and shape for implantation into the lesion after removal from the subject.

68. The method of claim 60, wherein the lesion is in at least one of the following tissues: bone, cartilage, ligament, tendon, heart, liver, kidney, brain, skin, cartilage, bladder, lung, thymus, thyroid, spinal cord, pancreas, skin, gut, bowel, blood vessels, bladder, joint cartilage, intervertebral disc, ligament, tendon, meniscus, or pancreas.

69. The method of claim 68, wherein the lesion is a bone lesion.

70. A method for producing a bone graft comprising contacting tissue with one or more bioactive agents ex vivo, wherein the bioactive agents stimulate at least a portion of the cells in the tissue to differentiate into bone cells, thereby producing a bone graft.

71. The method of claim 70, wherein the tissue is muscle tissue.

72. The method of claim 71, wherein the muscle tissue is skeletal muscle tissue.

73. The method of claim 70, wherein the tissue is fat tissue.

74. The method of claim 73, wherein the fat tissue is subcutaneous fat depot.

75. The method of claim 70, wherein bioactive agents are one or more of the following: at least one osteoinductive polypeptide and at least one nucleic acid encoding an osteoinductive polypeptide.

76. A method for producing a bone graft comprising contacting tissue ex vivo with at least one nucleic acid encoding at least one bioactive polypeptide thereby introducing said nucleic acid(s) into at least a portion of the cells in said tissue, wherein the bioactive agents are expressed in said cells, and wherein the bioactive agents stimulate at least a portion of the cells in the tissue to differentiate into bone cells, thereby forming a bone graft.

77. The method of claim 76, wherein the tissue is muscle tissue.

78. The method of claim 77, wherein the muscle tissue is skeletal muscle tissue.

79. The method of claim 76, wherein the tissue is fat tissue.

80. The method of claim 79, wherein the fat tissue is subcutaneous fat depot.

81. The method of claim 76, wherein bioactive agents are one or more osteoinductive polypeptides.

82. A composition comprising tissue and one or more bioactive molecules, wherein the bioactive agents stimulate at least a portion of the cells in the tissue to differentiate into bone cells.

83. The composition of claim 82, wherein the tissue is muscle tissue.

84. The composition of claim 83, wherein the muscle tissue is skeletal muscle tissue.

85. The composition of claim 82, wherein the tissue is fat tissue.

86. The composition of claim 85, wherein the fat tissue is subcutaneous fat depot.

87. The composition of claim 82, wherein the bioactive agents are one or more of the following: at least one osteoinductive polypeptide and at least one nucleic acid encoding an osteoinductive polypeptide.

88. A bone graft comprising muscle tissue which has been exposed to one or more bioactive agents, wherein the bioactive agents stimulate at least a portion of the cells in the muscle tissue to differentiate into bone cells.

89. A bone graft comprising fat tissue which has been exposed to one or more bioactive agents, wherein the bioactive agents stimulate at least a portion of the cells in the fat tissue to differentiate into bone cells.


SEQUENCE LISTING (4 sequences)

SEQ ID NO: 1 - DNA, 1547 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - DNA, 1878 bp, Homo sapiens
gggcgcagcggggcccgtctgcagcaagtgaccgacggccgggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatg

SEQ ID NO: 4 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH